(19) United States
(12) Patent Application Publication
(10) Pub. No.: US 2021/0208146 A1
(43) Pub. Date: Jul. 8, 2021

(54) METHODS FOR DETECTING PROSTATE CANCER PATHOLOGY ASSOCIATED WITH ADVERSE OUTCOMES

(71) Applicant: OPKO DIAGNOSTICS, LLC, Miami, FL (US)

(72) Inventors: DAVID OKRONGLY, Fort Lauderdale, FL (US); YAN DONG, Medford, MA (US)

(21) Appl. No.: 17/055,705

(22) PCT Filed: May 3, 2019
(86) PCT No.: PCT/US2019/030555

§ 371 Date: Nov. 16, 2020

Related U.S. Application Data
(60) Provisional application No. 62/672,336, filed on May 16, 2018.

(51) Int. Cl. G01N 33/574; G16B 5/20; G16H 10/40; G16H 50/30; G16H 20/40; G16H 50/70

(52) U.S. Cl. CPC G01N 33/57434; G16B 5/20; G16H 10/40; A61B 10/0241; G16H 20/40; G16H 50/70; G16H 50/30

(57) ABSTRACT

Aspects of the disclosure relate to improved methods and systems for predicting prostate cancer pathology associated with adverse outcomes in patients determined to a primary Gleason Grade of 3.

### RELATED APPLICATIONS

This application claims the benefit under 35 U.S.C. § 119(e) of U.S. Provisional Application No. 62/672,336, filed on May 16, 2018 which is hereby incorporated by reference herein in its entirety.

### TECHNICAL FIELD

Methods for detecting prostate cancer pathology associated with adverse outcomes and related compositions, systems, and kits associated therewith are generally described.

### BACKGROUND OF THE INVENTION

Over 1 million prostate biopsies are performed in the United States each year. Many of these biopsies will result in either overtreatment (e.g., in the form of radical prostatectomy) or under treatment due to inaccurate assessment of the risk of morbidity and mortality. Accordingly, improved methods for determining whether a patient has prostate cancer pathology associated with adverse outcomes are needed.

### SUMMARY OF THE INVENTION

The present disclosure is based, in part, on the finding that certain factors (e.g., protein marker levels and patient data) are differentially present in patients having prostate cancer pathology associated with adverse outcomes that present as having a primary Gleason score of 3 on biopsy.

Aspects of the disclosure relate to improved methods for predicting whether a subject having a primary Gleason score of 3 on biopsy has cancer pathology associated with adverse outcome (e.g., metastasis, mortality).

In one embodiment, an immunoassay-based method of evaluating a subject identified, based on a prostate tissue biopsy, as having prostate cancer characterized by a primary Gleason score of 3, is provided. The method comprises:

i) subjecting a blood sample of the subject to immunoassays that measure levels of total prostate specific antigen (tPSA), free prostate specific antigen (fPSA), intact prostate specific antigen (iPSA), and human kallikrein 2 (hK2); and

ii) determining a prostate cancer adverse outcome likelihood score for the subject based on the levels of fPSA, tPSA, iPSA, and hK2 and age of the subject.

In one embodiment, a method of treating a subject having prostate cancer, wherein the prostate cancer was characterized by a primary Gleason score of 3 based on a biopsy analysis, and wherein the prostate cancer was subsequently determined to be associated with an adverse outcome based on the levels of fPSA, tPSA, iPSA, and hK2 in the subject and age of the subject, is provided. The method comprises:

performing a radical prostatectomy procedure to remove the prostate tissue from the subject.

In one embodiment, a method of evaluating a treatment regimen for a subject identified as having prostate cancer characterized by a primary Gleason score of 3, is provided. The method comprises:

(i) obtaining information indicative of a level of total prostate specific antigen (tPSA) and free prostate specific antigen (fPSA), intact prostate specific antigen (iPSA), and human kallikrein 2 (hK2), in the subject;

(ii) determining a prostate cancer adverse outcome likelihood score for the subject based on the levels of fPSA, tPSA, iPSA, and hK2 and age of the subject; and

(iii) determining appropriateness of the treatment regimen based on the likelihood score.

In some embodiments with respect to a method described above and/or herein, the treatment regimen is active surveillance.

In some embodiments with respect to a method described above and/or herein, the treatment regimen comprises a chemotherapy, a radiation therapy, a surgical therapy, a cryotherapy, a hormone therapy, an immunotherapy, or a combination thereof.

In some embodiments with respect to a method described above and/or herein, the prostate cancer was identified as having a Gleason score of 3+4.

In some embodiments with respect to a method described above and/or herein, the measured levels of fPSA, tPSA, iPSA, and hK2 and age of the subject are weighted using a regression model.

In some embodiments with respect to a method described above and/or herein, determining the likelihood score comprises weighting a cubic spline term based on the measured tPSA level.

In some embodiments with respect to a method described above and/or herein, determining the likelihood score comprises weighting a cubic spline term based on the measured fPSA level.

In some embodiments with respect to a method described above and/or herein, the method further comprises removing at least a portion of the prostate of the subject, wherein the likelihood score is greater than a threshold value.

In some embodiments with respect to a method described above and/or herein, the method further comprises treating the subject, wherein treating the subject comprises a chemotherapy, a radiation therapy, a surgical therapy, a cryotherapy, a hormone therapy, an immunotherapy, or a combination thereof, and wherein the likelihood score is greater than a threshold value.

In some embodiments with respect to a method described above and/or herein, the method further comprises treating the subject with active surveillance, wherein the likelihood score is less than a threshold value.

In some embodiments with respect to a method described above and/or herein, the blood sample is obtained from the subject within 3 months from a biopsy.

In some embodiments with respect to a method described above and/or herein, step (i) and (ii) are performed within 3 months from a biopsy.

In some embodiments with respect to a method described above and/or herein, the method further comprises repeating steps (i).

In some embodiments with respect to a method described above and/or herein, the method further comprises repeating steps (i) and (ii) at least once a year for up to five years.

In some embodiments with respect to a method described above and/or herein, determining the likelihood score comprises weighting the measured levels of fPSA, tPSA, iPSA, and hK2.

In some embodiments with respect to a method described above and/or herein, the prostate cancer pathology associated with adverse outcomes has a pathological stage of at least T3b.

In one embodiment, a method for determining a probability of prostate cancer pathology associated with adverse outcomes is provided. The method comprises:

receiving, via an input interface, information indicative of a level of total prostate specific antigen (tPSA), free prostate specific antigen (fPSA), intact prostate specific antigen (iPSA), and human kallikrein 2 (hK2) in a subject and information indicative of age of a subject;

evaluating, using at least one processor, a logistic regression model based on the received information to determine a probability of prostate cancer pathology associated with adverse outcomes, wherein evaluating the logistic regression model consists essentially of:

determining the probability of prostate cancer pathology associated with adverse outcomes based on the information indicative of the level of tPSA, fPSA, iPSA, and hK2, and the information indicative of the subject's age; and

outputting an indication of the probability of prostate cancer pathology associated with adverse outcomes, wherein the subject has been identified as having Gleason 6 or 3+4 prostate cancer on biopsy.

In some embodiments with respect to a method described above and/or herein, the model outputs a risk score, wherein the output is indicative of prostate cancer pathology associated with adverse outcomes.

In some embodiments with respect to a method described above and/or herein, a risk score of less than 7.5% is indicative of low risk prostate cancer pathology associated with adverse outcomes.

In some embodiments with respect to a method described above and/or herein, a risk score of between 7.5% and 20% is indicative of intermediate risk prostate cancer pathology associated with adverse outcomes.

In some embodiments with respect to a method described above and/or herein, a risk score of greater than 20% is indicative of high risk prostate cancer pathology associated with adverse outcomes.

In some embodiments with respect to a method described above and/or herein, the subject is eligible for active surveillance.

In one embodiment, a computer for determining a probability of prostate cancer pathology associated with adverse outcomes is provided. The computer comprises:

an input interface configured to receive information indicative of a level of total prostate specific antigen (tPSA), free prostate specific antigen (fPSA), intact prostate specific antigen (iPSA), and human kallikrein 2 (hK2) in a subject and information indicative of a subject's age;

at least one processor programmed to evaluate a logistic regression model based, at least in part, on the received information to determine a probability of prostate cancer pathology associated with adverse outcomes, wherein evaluating the logistic regression model consists essentially of:

determining the probability of prostate cancer pathology associated with adverse outcomes, at least in part, on the information indicative of the level of tPSA, fPSA, iPSA, and hK2 and the information indicative of the subject's age; and

an output interface configured to output an indication of the probability of prostate cancer pathology associated with adverse outcomes,

wherein the subject has been identified as having Gleason 6 or 3+4 prostate cancer on biopsy.

In one embodiment, a system for determining a probability of prostate cancer pathology associated with adverse outcomes, the system comprises:

(a) a detector configured to measure a level of total prostate specific antigen (tPSA), free prostate specific antigen (fPSA), intact prostate specific antigen (iPSA), and human kallikrein 2 (hK2) in a subject; and

(b) a computer in electronic communication with the detector, wherein the computer comprises:
(i) an input interface configured to receive information indicative of a level of tPSA, fPSA, iPSA, and hK2 in the subject and information indicative of the subject's age;
(ii) at least one processor programmed to evaluate a logistic regression model based, at least in part, on the received information to determine a probability of prostate cancer pathology associated with adverse outcomes, wherein evaluating the logistic regression model consists essentially of:

determining the probability of prostate cancer pathology associated with adverse outcomes, at least in part, on the information indicative of the level of tPSA, fPSA, iPSA, and hK2 in the subject and information indicative of the subject's age; and
(iii) an output interface configured to output an indication of the probability of prostate cancer pathology associated with adverse outcomes,

wherein the subject has been identified as having Gleason 6 or 3+4 prostate cancer on biopsy.

In some embodiments with respect to a method, computer, or system described above and/or herein, a clinical stage of the biopsy is lower than T3.

In some embodiments with respect to a method, computer, or system described above and/or herein, the subject has been identified as having Gleason 6 and a tPSA level of less than 20 ng/mL. The method, computer, or system of any preceding claim, wherein the subject has been identified as having Gleason 3+4 and a tPSA level of less than 10 ng/mL.

In one embodiment, an immunoassay method comprises: i) subjecting a blood sample of a prostate cancer patient having a primary Gleason score of 3 and a Grade Group designation of 1 or 2 to immunoassays that measure levels of total prostate specific antigen (tPSA), free prostate specific antigen (fPSA), intact prostate specific antigen (iPSA), and human kallikrein 2 (hK2); and ii) determining a risk score predictive of an underlying adverse pathology associated with adverse outcomes in said prostate cancer patient based on the measured levels of fPSA, tPSA, iPSA, and hK2 and age of the subject wherein the risk score is provided in a percentage range of between 0-100% and further wherein a risk score of about 0-7.5% is predictive of a low risk of having an underlying adverse pathology; a risk score of about 7.5 to 20% is predictive of an elevated intermediate risk of having an underlying adverse pathology and a risk score of about 20% or greater is predictive of a high risk of having an underlying adverse pathology.

In some embodiments with respect to an immunoassay method described above and/or herein, the prostate cancer patient is on active surveillance as defined by NCCN and AUA.

In some embodiments with respect to an immunoassay method described above and/or herein, the underlying adverse pathology includes seminal vesicle or lymph node invasion.

In some embodiments with respect to a method described above and/or herein, the AUC of the method is greater than or equal to about 0.7, greater than or equal to about 0.72, or greater than or equal to about 0.74.

Another aspect of the present disclosure relates to the finding that certain factors (e.g., protein marker levels and patient data) are differentially present in patients having aggressive prostate cancer pathology that present as having a primary Gleason score of 3 on biopsy.

Certain aspects of the disclosure relate to improved methods for predicting whether a subject having a primary Gleason score of 3 on biopsy has cancer pathology associated with adverse outcome (e.g., metastasis, mortality).

In one embodiment, a method for determining a probability of at least pT3a prostate cancer is provided. The method comprises: subjecting a blood sample of the subject to immunoassays that measure levels of total prostate specific antigen (tPSA), free prostate specific antigen (fPSA), intact prostate specific antigen (iPSA), and human kallikrein 2 (hK2) in a subject; and determining the probability of at least pT3a prostate cancer by weighting the measured levels of fPSA, tPSA, iPSA, and hK2 and information indicative of at least one clinical factor of the subject, wherein the subject has been identified as having Gleason 6 or 3+4 prostate cancer on biopsy.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

The following drawings form part of the present specification and are included to further demonstrate certain aspects of the present disclosure, which can be better understood by reference to one or more of these drawings in combination with the detailed description of specific embodiments presented herein.

FIG. 1A is flowchart showing a process for determining whether a patient having a primary Gleason score of 3 on biopsy has prostate cancer pathology associated with adverse outcomes in accordance with some embodiments of the invention.

FIG. 1B is a schematic illustration of a computer configured for implementing a process for determining whether a patient having a primary Gleason score of 3 on biopsy has prostate cancer pathology associated with adverse outcomes in accordance with some embodiments of the invention.

FIG. 1C is a schematic of a computer network configured for implementing a process for determining whether a patient having a primary Gleason score of 3 on biopsy has prostate cancer pathology associated with adverse outcomes in accordance with some embodiments of the invention.

FIG. 2 shows a Kaplan-Meier graph used to compare biochemical recurrence rates between patients with kallikrein panel score lower than 20% and higher 20% in accordance with some embodiments of the invention.

### DETAILED DESCRIPTION OF THE INVENTION

Aspects of the disclosure relate to improved methods for predicting whether a subject having a primary Gleason score of 3 (e.g., Grade Group 1 or 2) on biopsy (i.e., “target subject” or “subject in the target population”) would have prostate cancer pathology associated with adverse outcomes (e.g., prostate cancer metastasis, mortality). Thus, methods disclosed herein may be employed by a healthcare provider for purposes of determining whether a subject in the target population can be safely monitored by Active Surveillance or should be considered for more extensive clinical evaluation and/or treatment.

The widespread use of prostate specific antigen (PSA) as the primary screening test for prostate cancer has dramatically increased the detection of clinically insignificant cancers. It is estimated that up to 60% of prostate cancer diagnosed via PSA screening has a low risk of causing morbidity or mortality. Nonetheless, many of these men undergo invasive treatment (e.g., surgery, radiotherapy) associated with significant risk of urinary or sexual dysfunction. Active Surveillance, in which patients having cancer with a very low, low, or favorable intermediate risk of an adverse outcome as determined, e.g., by biopsy are closely monitored for disease progression, has emerged to address the over-diagnosis and overtreatment of prostate cancer. However, the efficacy and long term safety of Active Surveillance depends on the ability to accurately distinguish prostate cancer having a low risk of morbidity and/or mortality from high risk prostate cancer. Recent studies have shown that certain widespread and standard indicators of high risk cancer (e.g., biopsy Gleason grade, Grade Group, various clinicopathologic features obtained at the time of prostate biopsy), and accordingly indicators for Active Surveillance or surgical and/or radiotherapy intervention, have limited prognostic value. As a result, a significant number of men still undergo unnecessary invasive treatments, such as radical prostatectomy (RP) or radiotherapy. Conversely, some men, who may be indicated as candidates to be safely monitored by Active Surveillance, may in fact have an underlying adverse pathology missed by the standard indicators. Accordingly, improved methods for better assessing the true risk of prostate cancer pathology associated with adverse outcomes are needed.

The present disclosure relates, in part, to the identification of prostate cancers associated with adverse outcomes and to the surprising discovery that certain subject data (e.g., age) and levels of certain protein markers can be used to detect these cancers. The methods, disclosed herein, may detect the relatively small number of subjects in the target population (i.e., population of subjects having prostate cancer with a primary Gleason grade of 3 on biopsy) with an underlying pathology associated with adverse outcomes that would only be revealed if the subject were to actually undergo RP. For instance, the methods may be used to determine the risk of a subject eligible for Active Surveillance (AS), accordingly to the American Urological Association (AUA) and/or National Comprehensive Cancer Network (NCCN) guidelines (e.g., very low, low, or favorable intermediate risk subjects), of having a prostate cancer pathology associated with adverse outcomes. Very low, low, or favorable intermediate risk subjects may be offered AS, but it is known that some subjects will harbor prostate cancer pathology associated with adverse outcomes, e.g., that is not detected via standard indicators and/or may only be detected by RP. AS utilizes various methods (e.g., frequent biopsy, MRI, molecular diagnostic tests) to detect disease progression or better define risk. However, if the prostate cancer pathology associated with adverse outcomes remains undetected and/or unsuspected on AS the subject may progress to metastatic disease or death. Some target subjects elect for overtreatment of their disease out of concern that standard indicators and/or AS may fail to detect true risk and/or disease progression. The present disclosure provides for improved methods for better assessing the true risk of prostate cancer pathology associated with adverse outcomes.

Prostate cancer pathology associated with adverse outcomes, as used herein, refers to prostate cancer that has a pathological stage of at least T3b (pT3b) and/or has a primary pathological Gleason grade at RP of greater than or equal to 4 (e.g., Grade Group 3 or higher). As used herein, pT3b has its ordinary meaning in the art and may refer to prostate cancer having seminal vesicle involvement. In some embodiments, a prostate cancer pathology associated with adverse outcomes has a pathological stage of at least pT3b and/or pathologic Grade Group 3 or higher. In some embodiments, a prostate cancer pathology associated with adverse outcomes has a pathological stage of at least T3b. In certain embodiments, a prostate cancer pathology associated with adverse outcomes has a pathologic Grade Group 3 or higher (e.g., Grade Group 3, Grade Group 4, Grade Group 5). In some embodiments, the adverse outcome is prostate cancer metastasis. In certain embodiments, the adverse outcome is prostate-cancer specific mortality. In embodiments in which the target subject has undergone a treatment for prostate cancer, the adverse outcome may be biochemical recurrence. For example, the method may be configured to predict the probability of a target subject, who has underwent radical prostatectomy, having biochemical recurrence at a later date.

As noted above, certain subject data (e.g., age) and levels of certain protein markers can be used to detect prostate cancer pathology associated with adverse outcomes in a subject. The subject may have been previously diagnosed with prostate cancer. For instance, at the time of biopsy, the subject may have been identified as having Grade Group 1 or Grade Group 2. The Grade Groups and their association with Gleason score are provided in the table below.

Gleason score = primary + secondary Gleason grades:

| | | |
|---|---|---|
| 3 + 3 = 6 | | Grade Group 1 |
| 3 + 4 = 7 | | Grade Group 2 |
| 4 + 3 = 7 | | Grade Group 3 |
| 4 + 4 = 8 | | |
| | {close oversize brace} | Grade Group 4 |
| 3 + 5 = 8 | | |
| 4 + 5 = 9 | | |
| | {close oversize brace} | Grade Group 5 |
| 5 + 5 = 10 | | |

In some embodiments, the target subject is eligible for AS according to NCCN and/or AUA guidelines. For example, the target subject may fall within the very low risk, low risk, or favorable intermediate risk groups provided in the table below.

| Risk group | Clinical/pathologic features |
|---|---|
| Very low^{g} | T1c AND |
| | Gleason score ≤6/grade group 1 AND |
| | PSA <10 ng/mL AND |
| | Fewer than 3 prostate biopsy fragments/cores |
| | positive, ≤50% cancer in each fragment/core^{h} AND |
| | PSA density <0.15 ng/mL/g |
| Low^{g} | T1-T2a AND |
| | Gleason score ≤6/grade group 1 AND |
| | PSA <10 ng/mL |
| Favorable | T2b-T2c OR |
| intermediate^{g} | Gleason score 3 + 4 = 7/grade group 2 OR |
| | PSA 10-20 ng/mL |
| | AND |
| | Percentage of positive biopsy cores <50% |

In one example, the methods described herein can be used to determine the likelihood of having prostate cancer pathology associated with adverse outcomes in a subject having prostate cancer in the very low risk group. In another example, the methods described herein can be used to determine the likelihood of having prostate cancer pathology associated with adverse outcomes in a subject having prostate cancer in the low risk group. As yet another example, the methods described herein can be used to determine the likelihood of having prostate cancer pathology associated with adverse outcomes in a subject having prostate cancer in the favorable intermediate risk group. In some embodiments, the methods described herein can be used to determine the likelihood of having prostate cancer pathology associated with adverse outcomes in a subject having prostate cancer that is not recommended for radical prostatectomy based on current guidelines (e.g., NCCN, AUA). In certain embodiments, the subject may already be on Active Surveillance.

In some embodiments, a method for detecting prostate cancer pathology associated with adverse outcomes in a subject involves subjecting a blood sample from the subject to immunoassays that measure levels of tPSA and free prostate specific antigen (fPSA) and at least two protein marker levels selected from the group consisting of intact prostate specific antigen (iPSA), human kallikrein 2 (hK2), pre-pro precursor prostate specific antigen (pre-pro PSA, or [−2] proPSA), Microseminoprotein, beta-(MSMB), and macrophage inhibitory cytokine-1 (MIC-1). In some embodiments, the method comprises subjecting a blood sample from the subject to immunoassays that measure levels of tPSA, fPSA, and two protein marker levels selected from the group consisting of iPSA, hK2, pre-pro PSA, MSMB, and MIC-1. In a particular embodiment, the method comprises subjecting a blood sample from the subject to immunoassays that measure levels of tPSA, fPSA, iPSA, and hK2. In certain embodiments, the method comprises subjecting a blood sample from the subject to immunoassays that measure levels of tPSA, fPSA, and three protein marker levels selected from the group consisting of iPSA, hK2, pre-pro PSA, MSMB, and MIC-1. In certain embodiments, the method comprises subjecting a blood sample from the subject to immunoassays that measure levels of tPSA, fPSA, and four protein marker levels selected from the group consisting of iPSA, hK2, pre-pro PSA, MSMB, and MIC-1. In some instances, the method comprises subjecting a blood sample from the subject to immunoassays that measure levels of tPSA, fPSA, iPSA, hK2, pre-pro PSA, MSMB, and MIC-1. In some embodiments, at least some of the protein markers (e.g., one, two, three, four or more, tPSA and fPSA) are measured in a single assay. In certain embodiments, two or more (e.g., three or more, four or more, five or more) protein markers are measured in different assays.

In some embodiments, the level of the protein markers and certain subject data can be used to determine the likelihood of prostate cancer pathology associated with adverse outcomes. It has been surprisingly found, within the context of certain embodiments, that certain non-clinical data (e.g., data not derived from a clinical procedure) and levels of certain protein markers can be used to accurately detect the presence of prostate cancer pathology associated with adverse outcomes in a subject having been diagnosed with Grade Group 1 or Grade Group 2 prostate cancer, as described herein. For instance, it has been unexpectedly found that the age of the subject and levels of tPSA, fPSA, iPSA, and hK2 can be used to non-invasively determine the likelihood of prostate cancer pathology with adverse outcome potential not detected by the prostate biopsy, but proven because these patients opted for removal of the prostate by RP, permitting complete pathological examination of the prostate. In some embodiments, the likelihood of the subject having prostate cancer pathology associated with adverse outcomes is determined solely based on subject data and levels of certain protein markers. For example, the likelihood of the subject having prostate cancer pathology associated with adverse outcomes may be determined using a predictive model (e.g., a logistic regression model) that only includes variables derived from the subject data and the levels of the protein markers. In addition, other clinicopathologic and imaging features (age, PSA level, digital rectal examination (DRE) status, prostate volume, PSA density, total tumor length in all prostate biopsy cores, number of tumor containing cores at prostate biopsy, percentage of tumor-containing cores found at prostate biopsy, maximum percentage of cancer in any prostate biopsy core, total tumor length of Gleason grade 4, percentage of Gleason grade 4 tumor, findings from multiparametric magnetic resonance imaging (mpMRI) of the prostate, racial group, single or multiple germline genetic variations and mutations (e.g., APC, ATM, BAP1, BARD1, BMPR1A, BRCA1, BRCA2, BRIP1, CDH1, CDK4*, CDKN2A(p14ARF), CDKN2A (p16INK4a), CHEK2, EPCAM, GREM1, MITF, MLH1, MSH2, MSH6, MUTYH, NBN, PALB2, PMS2, POLD1, POLE, PTEN, RAD51C, RAD51D, SMAD4, STK11, and TP5), expression profiles of various coding and non-coding RNA molecules obtained from the tumor directly or from a blood sample, androgen receptor gene rearrangement status (e.g., AR7) determined by analysis of the biopsy tumor tissue, circulating tumor cells, or cell-free DNA, and molecular imaging studies using imaging agents to contrast malignant tissue) may all be potentially combined with the protein markers to enhance performance of the logistic regression model.

In some embodiments, a predictive model (e.g., a logistic regression model) is provided that incorporates levels of tPSA and fPSA and at least two protein marker levels selected from the group consisting of iPSA, hK2, pre-pro PSA, MSMB, and MIC-1 to determine the likelihood of having prostate cancer pathology associated with adverse outcomes (e.g., metastasis, mortality) in a target subject. For instance, a predictive model (e.g., a logistic regression model) is provided that incorporates levels of tPSA, fPSA, iPSA, and hK2 to determine the likelihood that a target subject would have prostate cancer pathology associated with adverse outcomes (e.g., prostate cancer adverse outcome likelihood score) on radical prostatectomy). In some embodiments, the predictive model further comprises information (e.g., non-clinical information) regarding the subject, such as age and/or other cliniopathologic or imaging parameters described herein.

In some embodiments, the model outputs a risk score, wherein the output is indicative of the likelihood that a target subject has prostate cancer pathology associated with adverse outcomes. In some embodiments, the model outputs a risk score between 0% and 100%. A risk score of less than 7.5% may be associated with a low risk of prostate cancer pathology associated with adverse outcomes. In some such cases, the target subject may be recommended for AS. A risk score of between about 7.5% and 20% may be associated with an intermediate risk of prostate cancer pathology associated with adverse outcomes. In some such cases, the target subject may be recommended for less aggressive treatment, such as, e.g., antiandrogen therapy, immunotherapy, local treatment with cryotherapy or high-intensity focal ultrasound. Alternatively, the target subject may opt for more aggressive treatment. A risk score of greater 20% may be associated with an high risk of prostate cancer pathology associated with adverse outcomes. In such cases, the target subject may be recommended for more aggressive treatment, such as RP or radiation.

In some embodiments, the measured protein marker levels (e.g., kallikrein levels) are at or above a threshold level which is indicative of an increased likelihood that a prostate tissue sample obtained through RP contains prostate cancer pathology associated with adverse outcomes. In some embodiments, the measured protein marker levels are at or above a threshold level which is indicative of an increased likelihood the subject will have prostate cancer pathology associated with adverse outcomes at surgery. In certain embodiments, the measured protein marker levels are at or above a threshold level which is indicative of an increased likelihood the subject will have an adverse outcome associated with prostate cancer without treatment (e.g., chemotherapy, a radiation therapy, a surgical therapy, a cryotherapy, a hormone therapy, an immunotherapy, or a combination thereof).

In some embodiments, the measured protein marker levels (e.g., kallikrein levels) are at or above a threshold level that is indicative of an increased likelihood that the patient has a prostate cancer pathology associated with adverse outcomes. In some embodiments, the measured protein marker levels are at or above a threshold level which is indicative of an increased likelihood the subject will have prostate cancer pathology associated with adverse outcomes at surgery. In certain embodiments, the measured protein marker levels are at or above a threshold level which is indicative of an increased likelihood the subject will have an adverse outcome associated with prostate cancer without treatment (e.g., chemotherapy, a radiation therapy, a high-intensity focused ultrasound (HIFU) therapy, a surgical therapy such as RP, a cryotherapy, a hormone therapy, an immunotherapy, or a combination thereof).

In some embodiments, the measured protein marker levels (e.g., kallikrein levels) are below a threshold level that is indicative of an increased likelihood that the prostate tissue sample obtained through RP would not contain a prostate cancer pathology associated with adverse outcomes. These patients would be advised to select Active Surveillance, as they are unlikely to harbor underlying pathology associated with adverse outcome and can be safely monitored.

In some embodiments, the likelihood is further determined by a nomogram, where a patient eligible for active surveillance may weight one or more factors, such as, for example, one or more parameters indicative of the subject's risk for harboring underlying pathology associated with an adverse outcome. As another example, the one or more factors may be one or more parameters indicative of the race of subject and/or the family history of prostate cancer. In some embodiments, the one or more factors does not include factors derived from a clinical procedure (e.g., biopsy, DRE). For instance, the one or more factors are not selected from the group consisting of number of prostate tissue biopsies performed on the subject to date; results of prior prostate tissue biopsies performed on the subject to date; occurrence of any negative biopsy since an initial diagnosis of non-aggressive prostate cancer; occurrence of any negative biopsy within one-year prior to obtaining the blood sample; total number of biopsies since an initial diagnosis of non-aggressive prostate cancer; prostate volume on prior biopsy; number of positive cores on prior biopsy; percent positive cores on prior biopsy; cross-sectional area of cancer in biopsy core sections; maximum cross-sectional area of cancer in any biopsy core sections; PSA density; maximum percent of positive cores from any prior biopsy; and maximum number of positive cores from any prior biopsy.

In other embodiments, the one or more factors include factors derived from a clinical procedure (e.g., biopsy, DRE). For instance, the one or more factors may be selected from the group consisting of number of prostate tissue biopsies performed on the subject to date; results of prior prostate tissue biopsies performed on the subject to date; occurrence of any negative biopsy since an initial diagnosis of non-aggressive prostate cancer; occurrence of any negative biopsy within one-year prior to obtaining the blood sample; total number of biopsies since an initial diagnosis of non-aggressive prostate cancer; prostate volume on prior biopsy; number of positive cores on prior biopsy; percent positive cores on prior biopsy; cross-sectional area of cancer in biopsy core sections; maximum cross-sectional area of cancer in any biopsy core sections; PSA density; maximum percent of positive cores from any prior biopsy; and maximum number of positive cores from any prior biopsy

In some embodiments, the likelihood that the prostate harbors an underlying prostate cancer pathology associated with adverse outcomes (but only known through RP) is determined by weighting the measured levels of fPSA, iPSA, tPSA, and/or hK2. In some embodiments, the likelihood that the prostate tissue sample obtained through RP will contain prostate cancer pathology associated with adverse outcomes is an output of a logistic regression model that weights measured levels of fPSA, iPSA, tPSA, and/or hK2. In some embodiments, the likelihood is also based on weighting at least one factor, such as, for example, a parameter indicative of the subject's age.

Methods are provided herein for evaluating a treatment regimen for a subject having prostate cancer having a primary Gleason grade 3 based on biopsy and/or is thought to be eligible for Active Surveillance. Such methods may involve a physician or health care provider obtaining a blood sample from a subject and determining the likelihood that the underlying prostate pathology, only obtainable through RP, contains prostate cancer pathology associated with adverse outcomes, based at least in part, on measured levels of protein markers determined using the blood sample. The blood sample may be processed locally (e.g., within the same health care facility or business that the subject is being evaluated) or may be sent out to an external or third-party laboratory or facility for processing and analysis. In some embodiments, a treatment regimen such as radical prostatectomy, radiotherapy, antiandrogen, or ablation treatment may be the recommended course of action. In certain embodiments, the treatment regimen is Active Surveillance. In some such cases, evaluating Active Surveillance for a subject comprises determining whether a subject is not a candidate for a treatment, as described in more detail below. In some embodiments, the treatment regimen comprises a chemotherapy, a radiation therapy, a surgical therapy, a cryotherapy, a hormone therapy, an immunotherapy, or a combination thereof.

Methods are provided herein for determining whether a subject is a candidate for a radical prostatectomy (RP) and other treatments listed herein. Such methods may involve a physician or health care provider obtaining a blood sample from a subject and determining the likelihood that the prostate tissue sample obtained through RP contains prostate cancer pathology associated with adverse outcomes, at least in part, on measured levels of protein markers determined using the blood sample. The blood sample may be processed locally (e.g., within the same health care facility or business that the subject is being evaluated) or may be sent out to an external or third-party laboratory or facility for processing and analysis.

The physician or healthcare provider may determine whether the subject is a candidate for RP based on the likelihood that the prostate tissue sample obtained through RP will contain prostate cancer pathology associated with adverse outcomes. In some embodiments, a physician or healthcare provider may set a likelihood cut-off (threshold level) in which a RP will be indicated if a probability is at or above the cut-off. For example, if the probably is greater than 20% then the physician or healthcare provider may determine that the subject is a candidate for an aggressive treatment (e.g., RP).

In some embodiments, a physician or healthcare provider may set a likelihood range in which a RP will be indicated if a probability is within the range. For example, if the probability is within a range of 7.5 to 20%, then the physician or healthcare provider may determine that the subject is a candidate for less aggressive treatment.

In some embodiments, if a likelihood is below a cut-off (e.g., less than 7.5%) then a physician or healthcare provider will not order a RP but will continue to monitor the subject or recommend monitoring the subject, e.g., for increases in probability levels or changes in other risk factors indicative of prostate cancer. For example, the physician or healthcare provider continue on-going AS.

As a non-limiting set of examples, the physician or healthcare provider may monitor or recommend monitoring the subject every two months, six months, ten months, every year, every 1.5 years, every 2 years, every 2.5 years, every 3 years, every 3.5 years, every 4 years, every 4.5 years, or every 5 years.

In another aspect, the present disclosure relates, in part, to the identification of aggressive prostate cancers and to the surprising discovery that certain subject data (e.g., age, age, prostate volume, and total tumor length) and levels of certain protein markers can be used to detect these cancers. Aggressive prostate cancer, as used herein, refers to prostate cancer that has a pathological stage of at least T3a (pT3a). As used herein, pT3a refers to prostate cancer with extraprostatic (extracapsular) extension or microscopic invasion of the bladder neck. As noted above, certain subject data (e.g., age, prostate volume, and total tumor length) and levels of certain protein markers can be used to detect aggressive prostate cancer. The subject may have been previously diagnosed with prostate cancer as described above with respect to prostate cancer pathology associated with adverse outcomes (e.g., deemed to be=eligible for AS).

In some embodiments, a method for detecting aggressive prostate cancer in a subject involves subjecting a blood sample from the subject to immunoassays that measure levels of tPSA and fPSA and at least two protein marker levels selected from the group consisting of iPSA, hK2, pre-pro PSA, and MIC-1. In some embodiments, the method comprises subjecting a blood sample from the subject to immunoassays that measure levels of tPSA, fPSA, and two protein marker levels selected from the group consisting of iPSA, hK2, pre-pro PSA, MSMB, and MIC-1. In a particular embodiment, the method comprises subjecting a blood sample from the subject to immunoassays that measure levels of tPSA, fPSA, iPSA, and hK2. In certain embodiments, the method comprises subjecting a blood sample from the subject to immunoassays that measure levels of tPSA, fPSA, and three protein marker levels selected from the group consisting of iPSA, hK2, pre-pro PSA, and MIC-1. In some embodiments, the method comprises subjecting a blood sample from the subject to immunoassays that measure levels of tPSA, fPSA, and four protein marker levels selected from the group consisting of iPSA, hK2, pre-pro PSA, and MIC-1. In some instances, the method comprises subjecting a blood sample from the subject to immunoassays that measure levels of tPSA, fPSA, iPSA, hK2, pre-pro PSA, MSMB, and MIC-1. In some embodiments, at least some of the protein markers (e.g., one, two, three, four or more, tPSA and fPSA) are measured in a single assay. In certain embodiments, two or more (e.g., three or more, four or more, five or more) protein biomarkers are measured in different assays.

It has been surprisingly found, within the context of certain embodiments, that certain subject data and levels of certain protein markers can be used to accurately detect aggressive prostate cancer in a subject having prostate cancer, as described herein. For instance, it has been unexpectedly found that the age, prostate volume, and total tumor length of the subject and levels of tPSA, fPSA, iPSA, and hK2 can be used to determine the likelihood of aggressive prostate cancer. In some embodiments, the likelihood of the subject having aggressive prostate cancer is determined solely based on subject data and levels of certain protein markers. For example, the likelihood of the subject having aggressive prostate cancer is determined using a predictive model (e.g., a logistic regression model) that only includes variables derived from the subject data and the levels of the protein markers.

In some embodiments, a predictive model (e.g., a logistic regression model) is provided that incorporates levels of tPSA and fPSA and at least two protein marker levels selected from the group consisting of iPSA, hK2, pre-pro PSA, and MIC-1 to determine the likelihood of having prostate cancer pathology associated with adverse outcomes (e.g., biochemical recurrence, metastasis, mortality). For instance, a predictive model (e.g., a logistic regression model) is provided that incorporates levels of tPSA, fPSA, iPSA, and hK2 to determine the likelihood that a subject having low-grade score cancer on biopsy would have aggressive prostate cancer on radical prostatectomy. In some embodiments, the predictive model further comprises information regarding the subject, such as age, prostate volume, and/or total tumor length.

The physician or healthcare provider may determine whether the subject is a candidate for RP based on the likelihood that the prostate tissue sample obtained through RP will contain aggressive prostate cancer. In some embodiments, a physician or healthcare provider may set a likelihood cut-off (threshold level) in which a RP will be indicated if a probability is at or above the cut-off. For example, if the probably is greater than 20% then the physician or healthcare provider may determine that the subject is a candidate for an aggressive treatment (e.g., RP).

In some embodiments, a physician or healthcare provider may set a likelihood range in which a RP will be indicated if a probability is within the range. For example, if the probability is within a range of 7.5 to 20%, then the physician or healthcare provider may determine that the subject is a candidate for less aggressive treatment.

In some embodiments, if a likelihood is below a cut-off (e.g., less than 7.5%) then a physician or healthcare provider will not order a RP but will continue to monitor the subject or recommend monitoring the subject, e.g., for increases in probability levels or changes in other risk factors indicative of prostate cancer. For example, the physician or healthcare provider continue on-going AS.

In some embodiments, the measured protein marker levels (e.g., kallikrein levels) are at or above a threshold level which is indicative of an increased likelihood that a prostate tissue sample obtained through RP contains aggressive prostate cancer. In some embodiments, the measured protein marker levels are at or above a threshold level which is indicative of an increased likelihood the subject will have aggressive prostate cancer at surgery. The thresholds may be the same as those described herein with respect to prostate cancer pathology associated with adverse outcomes.

In some embodiments, the measured protein marker levels (e.g., kallikrein levels) are below a threshold level which is indicative of an increased likelihood that the prostate tissue sample obtained through RP does not contain aggressive prostate cancer. In some embodiments, the measured protein marker levels are below a threshold level which is indicative of an increased likelihood the subject will not have aggressive prostate cancer at surgery.

In some embodiments, the likelihood is further determined by weighting one or more factors, such as, for example, one or more parameters indicative of the subject's age prostate volume, and/or total tumor length.

In some embodiments, the likelihood that the prostate tissue sample obtained through RP will contain aggressive prostate cancer is determined by weighting the measured levels of fPSA, iPSA, tPSA, and/or hK2. In some embodiments, the likelihood that the prostate tissue sample obtained through RP will contain aggressive prostate cancer is an output of a logistic regression model that weights measured levels of fPSA, iPSA, tPSA, and/or hK2. In some embodiments, the likelihood is also based on weighting at least one factor, such as, for example, a parameter indicative of the subject's age, prostate volume, and/or total tumor length.

### Analysis of Biological Samples

Any sample that may contain prostate cancer cells (e.g., prostate tissue sample) can be analyzed by the assay methods described herein. Any sample that may contain markers of prostate cancer cells (e.g., one or more kallikrein markers) can be analyzed by the assay methods described herein. The methods described herein may involve providing a sample obtained from a subject. In some embodiments, the methods described herein may involve procuring a sample from a subject. In some examples, the sample to be analyzed by the assay methods is a biological sample.

As used herein, a “biological sample” refers to a composition that comprises tissue, e.g., whole blood, blood plasma, serum, or protein, from a subject. A biological sample may include both an initial unprocessed sample taken from a subject as well as subsequently processed, e.g., partially purified or preserved forms of a sample taken from a subject. Exemplary samples include blood (including whole blood, blood plasma, or serum), tears, or mucus. In some embodiments, the sample is a body fluid sample such as a serum or blood plasma sample. In certain embodiments, the sample is a whole blood sample. In some embodiments, multiple (e.g., at least 2, 3, 4, 5, or more) biological samples may be collected from a subject, over time or at particular time intervals. These multiple samples may be used, for example, to assess disease progression over time or to evaluate the efficacy of a treatment.

A biological sample can be obtained from a subject using any means known in the art. In some embodiments, the sample is obtained from the subject by removing the sample (e.g., a prostate tissue sample) from the subject. In some embodiments, the sample is obtained from the subject by a surgical procedure (e.g., radical prostatectomy). In some embodiments, the sample is obtained from the subject by a biopsy (e.g., a prostate biopsy). Examples of a prostate biopsy include, but are not limited to, a transrectal ultrasound (TRUS)-guided systematic biopsy of the prostate, a transurethral biopsy, a transperineal prostate biopsy, and a MRI-guided prostate biopsy.

In some embodiments, more than one sample is obtained from the same patient (e.g., a blood sample and a prostate biopsy sample). In some embodiments, the blood sample and prostate biopsy sample are obtained on the same day. In some embodiments, the prostate biopsy sample is obtained before the blood sample is obtained. In some embodiments, the blood sample is obtained before the prostate biopsy sample is obtained. In certain embodiments, more than one blood sample is obtained. In some embodiments, a first blood sample may be obtained before the prostate biopsy sample, and a second blood sample may be obtained after the prostate biopsy sample. In some embodiments, a blood sample is obtained within about 1, 2, 3, 4, 5, 6, 7, or 8 days of a prostate biopsy sample. In some embodiments, a blood sample is obtained within about 1, 2, 3, 4, 5, 6, 7, or 8 weeks of a prostate biopsy sample. In certain embodiments, a blood sample is obtained within about 1, 2, 3, 4, 5, 6, 7, 8, 9, 10, 11, or 12 months of a biopsy sample. In some embodiments, a blood sample is obtained from a subject about every 1, 2, 3, 4, 5, 6, 7, 8, 9, 10, 11, or 12 months; or every 1, 2, 3, 4, 5, 6, or 7 years. In certain embodiments, a blood sample is obtained from a subject at least once within 3-9 months, at least once within 4-10 months, at least once within 5-11 months, or at least once within 6-12 months. In certain embodiments, a blood sample is obtained from a subject at least once per year for 2, 3, 4, 5, 6, 7, 8, 9, or 10 years.

In some embodiments, a prostate tissue sample may be characterized based on its clinical stage of cancer. In some embodiments, the prostate tissue sample may be characterized based on a Gleason grade. In some embodiments, the prostate tissue sample may be characterized based on a tumor, node, metastasis (TNM) system.

In some embodiments, a biological sample may be analyzed for multiple protein marker levels (e.g., levels of four or more of tPSA, fPSA, iPSA, hK2, pre-pro PSA, MIC-1). In certain embodiments, a biological sample may be analyzed for multiple kallikrein marker levels (e.g., levels of two or more of tPSA, fPSA, iPSA, and hK2). In some embodiments, multiple kallikrein marker levels are determined in parallel in the same assay (e.g., in a multiplex assay). In other embodiments, such antigen levels are determined in separate assays. In some embodiments, antigen levels are determined from the same original blood draw (e.g., a venous blood draw) from a subject. In some embodiments, antigen levels are determined from different blood draws. In some embodiments, antigen levels are determined using blood preparations from the same or different blood draws. In some embodiments, one or more antigen levels are determined using a blood preparation and one or more other antigens are determined using a different type of blood preparation, e.g., serum or whole blood. Blood plasma is a pale-yellow liquid component of blood. In some embodiments, blood plasma may be prepared by spinning a tube of blood containing an anticoagulant (e.g., Heparin, EDTA, etc.) in a centrifuge until blood cells and debris move to the bottom of the tube, after which the blood plasma may be poured or drawn off.

Diagnostic and/or Prognostic Applications

The levels of multiple protein markers (e.g., tPSA, fPSA, iPSA, and/or hK2) in a biological sample derived from a subject, determined as described herein, can be used for various clinical purposes, for example, identifying a subject as likely to have prostate cancer pathology associated with adverse outcomes and/or aggressive prostate cancer, identifying subjects suitable for a particular treatment (e.g., radical prostatectomy), and/or predicting likelihood of prostate cancer pathology associated with adverse outcomes or aggressive prostate cancer. Accordingly, described herein are diagnostic and prognostic methods for prostate cancer, for example, prostate cancer pathology associated with adverse outcomes, based on the levels of multiple protein markers (e.g., kallikreins).

The terms “subject” or “patient” may be used interchangeably and refer to a subject who needs the analysis as described herein. In some embodiments, the subject is a human or a non-human mammal. In some embodiments, the subject is suspected of or is at risk for prostate cancer. In some embodiments, the subject has prostate cancer. In some embodiments, the subject is suspected of or is at risk for high-grade prostate cancer. Examples of prostate cancer include, without limitation, acinar adenocarcinoma, ductal adenocarcinoma, transitional cell (or urothelial) prostate cancer, squamous cell prostate cancer, and small cell prostate cancer.

In some embodiments, the subject is a human patient having one or more symptom of a prostate cancer. For example, the subject may have problems urinating, blood in the urine or semen, erectile dysfunction, pain, weakness or numbness, loss of bladder or bowel control, or a combination thereof. In some embodiments, the subject has a symptom of prostate cancer, has a history of a symptom of prostate cancer, or has a history of low-grade prostate cancer. In some embodiments, the subject has more than one symptom of prostate cancer or has a history of more than one symptoms of prostate cancer. In some embodiments, the subject has no symptom of prostate cancer, has no history of a symptom of prostate cancer, or has no history of prostate cancer. In yet other embodiments, the subject is at risk for having an upgrade in prostate cancer.

Such a subject may exhibit one or more symptoms associated with the prostate cancer. Alternatively or in addition, such a subject may have one or more risk factors for prostate cancer, for example, an environmental factor associated with prostate cancer (e.g., geographic location), a family history of prostate cancer, or a genetic predisposition to developing prostate cancer.

Alternatively, the subject who needs the analysis described herein may be a patient having prostate cancer or suspected of having prostate cancer. Such a subject may currently be having a relapse, or may have suffered from the disease in the past (e.g., may be currently relapse-free), or may have low-grade prostate cancer. In some examples, the subject is a human patient who may be on a treatment (i.e., the subject may be receiving treatment) for the disease including, for example, a treatment involving chemotherapy or radiation therapy. In other instances, such a human patient may be free of such a treatment.

### Methods for Assaying Levels of Prostate Specific Antigens

Levels of prostate specific antigens (e.g., kallikrein markers: tPSA, iPSA, fPSA, and/or hK2) can be assessed by any appropriate method. In some embodiments, antibodies or antigen-binding fragments are provided that are suited for use in immunoassays. Such immunoassays may be competitive or non-competitive immunoassays in either a direct or indirect format.

Examples of an immunoassay that may be used in accordance with the methods described herein include, but are not limited to, an Enzyme Linked Immunoassay (ELISA), a radioimmunoassay (RIA), a sandwich assay (immunometric assay), a Sangia assay (silver amplified NeoGold immunoassay), a flow cytometry assay, a western blot assay, an immunoprecipitation assay, an immunohistochemistry assay, an immune-microscopy assay, a lateral flow immuno-chromatographic assay, and a proteomics array.

Antigens, antibodies, and/or antigen-binding fragments can be immobilized, e.g., by binding to solid supports (e.g., carriers, membrane, columns, proteomics array, etc.). Examples of solid support materials include, but are not limited to: glass, polystyrene, polyvinyl chloride, polyvinylidene difluoride, polypropylene, polyethylene, polycarbonate, dextran, nylon, amyloses, natural and modified celluloses, such as nitrocellulose, polyacrylamides, agaroses, and/or magnetite. One or more than one solid support material may be used in the solid supports, and may contain at least one solid material listed above. The nature of the solid support can be either fixed or suspended in a solution (e.g., beads, porous material, or a membrane).

In some embodiments, labeled antibodies or antigen binding fragments may be used as tracers to detect antigen bound antibody complexes. Examples of the types of labels which can be used to generate tracers include, but are not limited to: enzymes, radioisotopes, colloidal metals, fluorescent compounds (including time-resolved fluorescence), magnetic, chemiluminescent compounds, electrochemiluminescent compounds, and bioluminescent compounds. Radiolabeled antibodies are prepared in known ways by coupling a radioactive isotope such as ¹⁵³Eu, ³H, ³²P, ³⁵S, ⁵⁹Fe, or ¹²⁵I, which can then be detected by gamma counter, scintillation counter, and/or by autoradiography. As discussed herein, antibodies and antigen-binding fragments may alternatively be labeled with enzymes such as yeast alcohol dehydrogenase, horseradish peroxidase, alkaline phosphatase, and the like, then developed and detected spectrophotometrically or visually. Suitable fluorescent labels include fluorescein, isothiocyanate, fluorescamine, rhodamine, and the like, or complexes (chelates) of lanthanides salts (such as europium, terbium, samarium or dysprosium) with appropriate ligands to improve fluorescence. Suitable chemiluminescent labels may include luminol, imidazole, oxalate ester, luciferin, and others. Suitable electrochemiluminescent labels may include Ru(bpy)₃²⁺ (short for Tris(2,2′-bipyridyl)ruthenium(II)-complex), and others.

An immunoassay may comprise contacting the sample, e.g., a blood plasma sample, containing an antigen with an antibody, or antigen-binding fragment (e.g., F(ab), F(ab)₂), under conditions enabling the formation of binding complexes between antibody or antigen-binding fragment and antigen. In some embodiments, a plasma sample is contacted with an antibody or antigen-binding fragment under conditions suitable for binding of the antibody or antigen-binding fragment to a target antigen, if the antigen is present in the sample. This may be performed in a suitable reaction chamber, such as a tube, plate well, microchannel, membrane bath, cell culture dish, microscope slide, and/or other chamber. In some embodiments, an antibody or antigen-binding fragment is immobilized on a solid support. In some embodiments, the solid support (i.e., beads) can be further captured onto the surface of an electrode for obtaining an electrochemiluminescent signal.

An antibody or antigen binding fragment that binds to an antigen in a sample may be referred to as a capture antibody. In some embodiments, the capture antibody comprises a tag (e.g., a biotin label) that facilitates its immobilization to a solid support by an interaction involving the tag (e.g., a biotin-streptavidin interaction in which the streptavidin is immobilized to a solid support). In some embodiments, the solid support is the surface of a reaction chamber. In some embodiments, the solid support is of a polymeric membrane (e.g., nitrocellulose strip, Polyvinylidene Difluoride (PVDF) membrane, etc.) or inorganic material (glass, quartz, nitride or oxide of silicon, titanium, indium, tin or other elements). In some embodiments, the solid support is suspension of beads (e.g., plain beads or beads with a magnetic core). In other embodiments, the solid support is a biological structure (e.g., bacterial cell surface). Other exemplary solid supports are disclosed herein and will be apparent to one of ordinary skill in the art.

In some embodiments, the antibody or antigen-binding fragment is immobilized on the solid support prior to contact with the antigen. In other embodiments, immobilization of the antibody or antigen-binding fragment is performed after formation of binding complexes between the antibody or antigen binding fragment and the antigen. In still other embodiments, an antigen is immobilized on a solid support prior to formation of binding complexes between the antigen and the antibody or antigen-binding fragment. In some embodiments, a tracer may be added to the reaction chamber to detect immobilized binding complexes. In some embodiments, the tracer comprises a detectably labeled secondary antibody directed against the antigen. In some embodiments, the tracer comprises a detectably labeled secondary antibody directed against the capture antibody. In some embodiments, the primary antibody or antigen-binding fragment is itself detectably labeled.

In one embodiment, immunoassay methods disclosed herein comprise immobilizing antibodies or antigen-binding fragments to a solid support; applying a sample (e.g., a plasma sample) to the solid support under conditions that permit binding of antigen to the antibodies or antigen-binding fragments, if present in the sample; removing the excess sample from the solid support; applying a tracer (e.g., detectably labeled antibodies or antigen-binding fragments) under conditions that permit binding of the tracer to the antigen-bound immobilized antibodies or antigen-binding fragments; washing the solid support and assaying for the presence of the tracer.

In some embodiments, the antibody or antigen-binding fragment is immobilized on the solid support after contact with the antigen in a reaction chamber. In some embodiments, the antibody or antigen-binding fragment is immobilized on the solid support prior to contact with the antigen in a reaction chamber. In either case, a tracer may be added to the reaction chamber to detect immobilized binding complexes. In some embodiments, a tracer comprises a detectably labeled secondary antibody directed against the antigen. In some embodiments, the tracer comprises a detectably labeled secondary antibody directed against the primary antibody or antigen-binding fragment. As disclosed herein, the detectable label may be, for example, a radioisotope, a fluorophore, a luminescent molecule, an enzyme, a biotin-moiety, an epitope tag, or a dye molecule. Other suitable detectable labels are described herein.

In some embodiments, it has been found that performing certain immunoassays in low pH buffer leads to more sensitive antigen detection. Accordingly, in some embodiments, a tracer antibody is contacted with a capture antibody in a buffer having a pH in a range of 6.5 to less than 7.75 such that the tracer binds to the capture antibody-antigen complex. In some embodiments, a tracer antibody is contacted with a capture antigen-binding fragment in a buffer having a pH in a range of 6.5 to less than 7.75 such that the tracer binds to the capture antigen-binding fragment-antigen complex. In some embodiments, the buffer pH is about 6.5, 6.6, 6.7, 6.8, 6.9, 7.0, 7.1, 7.2, 7.3, 7.4, 7.5, or 7.6.

It should be appreciated that in any of the assays disclosed herein capture antibodies may be swapped with tracer antibodies.

In some embodiments, an immunoassay that measures the level of fPSA involves contacting fPSA present in the plasma blood sample with a capture antibody specific for fPSA or tPSA under conditions in which the first capture antibody binds to fPSA or tPSA, thereby producing a capture-antibody-PSA complex; and detecting the capture-antibody-PSA complex using a tracer specific for fPSA or tPSA. In some embodiments, the immunoassay comprises at least one capture antibody specific for fPSA or at least one tracer specific for fPSA. In some embodiments, the immunoassay comprises at least one capture antibody specific for fPSA and at least one tracer specific for fPSA. The capture antibody may be a H117 antibody. In some embodiments, the tracer comprises a 5A10 antibody or fragment thereof (e.g., a F(ab) fragment).

In some embodiments, an immunoassay that measures the level of iPSA involves contacting iPSA present in the plasma blood sample with a capture antibody specific for free PSA (which includes iPSA and nicked PSA) or free PSA, under conditions in which the second capture antibody binds at least to iPSA, thereby producing a capture-antibody-PSA complex and detecting the capture-antibody-PSA complex using a second tracer. In some embodiments, the tracer comprises a 4D4 antibody. In some embodiments, the immunoassay comprises at least one capture antibody specific for intact PSA or at least one tracer specific for intact PSA. In some embodiments, the immunoassay comprises at least one capture antibody specific for intact PSA and at least one tracer specific for intact PSA. In some embodiments, the capture antibody is a 5A10 antibody or fragment thereof (e.g., a F(ab) fragment).

In some embodiments, an immunoassay that measures the level of tPSA involves contacting tPSA present in the plasma blood sample with a capture antibody specific for tPSA under conditions in which the third capture antibody binds to tPSA, thereby producing a capture-antibody-tPSA complex; and detecting the capture-antibody-tPSA complex using a third tracer. In some embodiments the selectivity of the capture and third tracer antibodies result in an equimolar detection of free PSA and PSA complexed with alpha 1-antichymotrypsin (PSA-ACT). Equimolar detection means that the molar recovery of free PSA is within 5%, 10%, 20% or 30% of the molar recovery of PSA-ACT. In some embodiments, the tracer comprises a H50 antibody. In some embodiments, the capture antibody is a H117 antibody.

In some embodiments, an immunoassay that measures the level of hK2 involves contacting PSA in the plasma blood sample with blocking antibodies specific for PSA; contacting hK2 present in the plasma blood sample with a fourth capture antibody specific for hK2 under conditions in which the fourth capture antibody binds to hK2, thereby producing a capture-antibody-hK2 complex; and detecting the capture-antibody-hK2 complex using a fourth tracer. In some embodiments one or both of the fourth capture antibody and the fourth tracer may also be capable of binding to PSA. In some embodiments, the fourth capture antibody is capable of binding PSA and the fourth tracer is capable of binding to PSA. In some embodiments, the fourth capture antibody is incapable of binding PSA and the fourth tracer is capable of binding to PSA. In some embodiments, the fourth capture antibody is capable of binding PSA and the fourth tracer is incapable of binding to PSA. In some embodiments, the tracer comprises a 7G1 antibody. In some embodiments, the capture antibody is a 6H10 F(ab)₂. In some embodiments, the blocking antibodies comprise a 5H7 antibody, a 5H6 antibody, and a 2E9 antibody.

Table 1 below lists antibodies and antigen-binding fragments that may be used in the methods disclosed herein and their corresponding epitopes.

TABLE 1
Antibodies and Epitopes/Sources of Antibodies.

| Antibody Name | Epitope | Reference or Source |
|---|---|---|
| F(ab)₂ 6H10 | | Becker et al. 2000. Sensitive and Specific Immunodetection of |
| | | Human Glandular Kallikrein 2 in Serum. Clin Chem. 46(2), |
| | | 198-206. |
| 2E9 | amino acids 79-93 | Lilja et al. 1991. Prostate-Specific Antigen in Serum Occurs |
| | and/or 80-91 of | Predominantly in Complex with alpha-1-Antichymotrypsin. Clin |
| | PSA protein | Chem. 37(9), 1618-1625. Piironen, et al. Determination and |
| | | analysis of antigenic epitopes of prostate specific antigen (PSA) |
| | | and human glandular kallikrein 2 (hK2) using synthetic peptides |
| | | and computer modeling. Protein Science (1998), 7: 259-269 |
| 5F7 | | Nurmikko et al. 2000. Production and Characterization of |
| | | Novel Anti-Prostate-specific Antigen (PSA) Monoclonal |
| | | Antibodies That Do Not Detect Internally Cleaved Lys145- |
| | | Lys146 Inactive PSA. Clin Chem. 46(10): 1610-1618. |
| 5H6 | amino acids 225-237 | Nurmikko et al. 2000. Supra |
| | of PSA protein | |
| 7G1 | | Nurmikko et al. 2000. Supra |
| Fab 5A10 | amino acids 75-89, | Eriksson et al. 2000. Dual-label time-resolved |
| | 80-94 and/or 82-39 | immunofluorometric assay of free and total Prostate-specific |
| | of PSA protein | Antigen Based on Recombinant Fab Fragments. Clin Chem |
| | | 46(5), 658-666. |
| | | Piironen et al. Supra |
| 4D4 | amino acids 130-144 | U.S. Pat. No. 7,872,104 |
| | of PSA protein | |
| H117 | | U.S. Pat. No. 5,672,480 |
| H50 | | U.S. Pat. No. 5,672,480 |
| 5A10 | amino acids 75-89, | U.S. Pat. No. 5,939,533, European Collection of Animal Cell |
| | 80-94 and/or 82-39 | Cultures Accession number 93091201. |
| | of PSA protein | Piironen et al. Supra |

### Fluidic Sample Analyzers

It should be appreciated that any of the immunoassay methods disclosed herein may be performed or implemented using a fluidic device (e.g., a microfluidic device or a cassette) and/or a fluidic sample analyzer (e.g., a microfluidic sample analyzer). For example, a fluidic device (e.g., a microfluidic device) may be used to determine one or more characteristics of protein markers, such as kallikrein markers (e.g., levels of tPSA, fPSA, iPSA, and/or hK2). In some embodiments, a system may include a fluidic sample analyzer (e.g., a microfluidic sample analyzer) which, for example, may be configured to analyze a sample provided in a device (e.g., a cassette) having one or more fluidic channels (e.g., microfluidic channels) for containing and/or directing flow of a sample that comprises immunoassay components (e.g., antigen-antibody complexes, tracers, etc.). In some embodiments, an analyzer comprises an optical system including one or more light sources and/or one or more detectors configured for measuring levels of antigen-antibody complexes and/or tracers present in one or more fluidic channels (e.g., microfluidic channels). Furthermore, in some embodiments, systems are provided which may include a processor or computer programmed to evaluate a predictive model (e.g., a logistic regression model) in electronic communication with a fluidic device (e.g., a microfluidic device) and/or a fluidic sample analyzer (e.g., a microfluidic sample analyzer) or other device for determining a probability of an event associated with prostate cancer based on levels of markers (e.g., levels of tPSA, fPSA, iPSA, and/or hK2).

In one particular example, a system includes a fluidic sample analyzer (e.g., a microfluidic sample analyzer) comprising a housing and an opening in the housing configured to receive a device (e.g., a cassette) having at least one fluidic channel (e.g., a microfluidic channel), wherein the housing includes a component configured to interface with a mating component on the device to detect the device within the housing. The system also includes a pressure-control system positioned within the housing, the pressure-control system configured to pressurize the at least one fluidic channel (e.g., a microfluidic channel) in the device to move the sample through the at least one fluidic channel (e.g., a microfluidic channel). The system further includes an optical system positioned within the housing, the optical system including at least one light source and at least one detector spaced apart from the light source, wherein the light source is configured to pass light through the device when the device is inserted into the sample analyzer and wherein the detector is positioned opposite the light source to detect the amount of light that passes through the cassette. The system may include a user interface associated with the housing for inputting at least one clinical factor (e.g., the age of a person). The system may include a processor in electronic communication with the fluidic sample analyzer (e.g., a microfluidic sample analyzer), the processor programmed to evaluate a logistic regression model as described herein in combination with information indicative of levels of one or more protein (e.g., kallikrein) markers selected from: tPSA and fPSA and at least two selected from the group consisting of iPSA, pre-pro PSA, MIC-1, and hK2 in a blood sample of a subject previously diagnosed as having a low-grade score prostate cancer.

Non-limiting examples of suitable fluidic devices are disclosed in U.S. Patent Application Publication Number U.S. 2013/0273643, entitled “METHODS AND APPARATUSES FOR PREDICTING RISK OF PROSTATE CANCER AND PROSTATE GLAND VOLUME,” which published on Oct. 17, 2013, and U.S. Pat. No. 8,765,062, entitled “Systems and Devices for Analysis of Samples”, which issued on Jul. 1, 2014, the contents of which are incorporated herein by reference in their entirety for all purposes. It should be appreciated, however, that other types of device may also be used (e.g., plate readers, analyzers for microwell ELISA-type assays, etc.) as the disclosure is not limited in this respect.

### Predictive Models and Computer Implemented Methods

Aspects of the disclosure provide computer implemented methods for determining the likelihood that a prostate tissue sample obtained from the subject through radical prostatectomy would contain prostate cancer pathology associated with adverse outcomes.

Such methods may involve receiving, via an input interface, information indicative of the level of protein markers (e.g., tPSA, fPSA, iPSA, and/or hK2) present in a sample (e.g., a blood sample) of a subject and receiving, via an input interface, patient information, such as information relating to the subject's age. In some embodiments, the methods further involve evaluating, using at least one processor, a suitable predictive model (e.g., a logistic regression model) based, at least in part, on the received information to determine a likelihood of a prostate cancer pathology associated with adverse outcomes. The predictive model may generate the likelihood of prostate cancer pathology associated with adverse outcomes based, at least in part, on measured levels of tPSA, fPSA, iPSA, and/or hK2 and patient information, such as information relating to the subject's age.

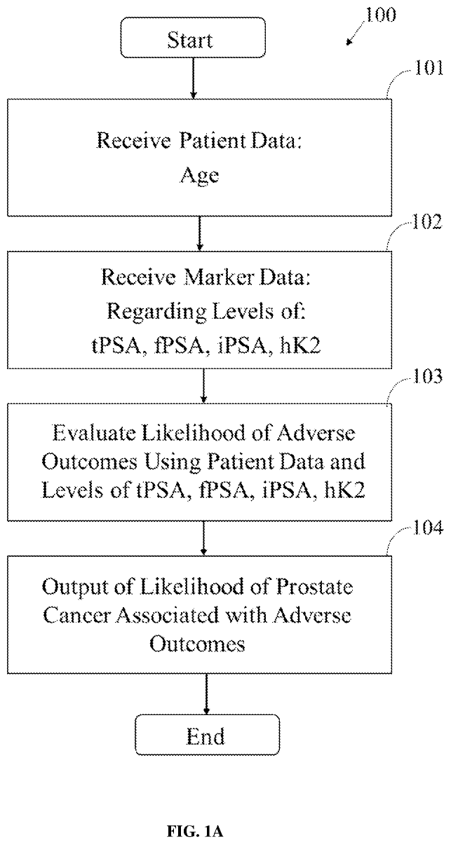

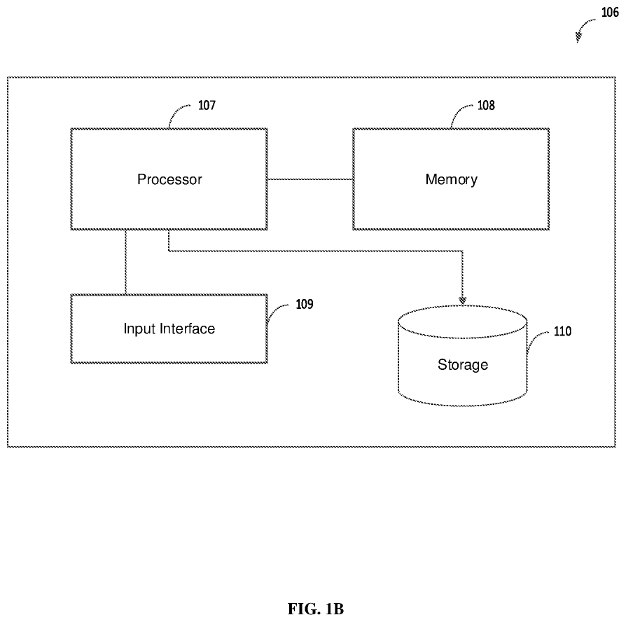

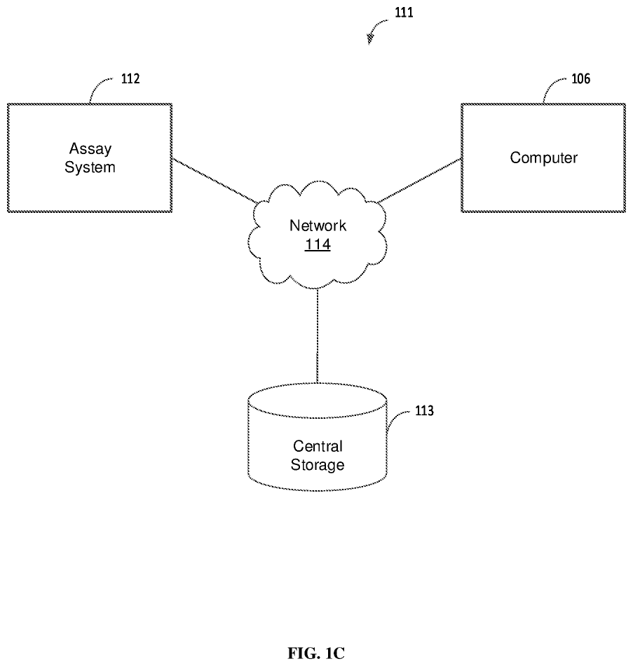

FIG. 1A shows a flowchart of a process 100 in accordance with some embodiments of the disclosure. In step 101, one or more values representing patient data corresponding to age are received by at least one processor for processing using one or more of the techniques described herein. In step 102 one or more values representing marker data for protein markers (e.g., tPSA, fPSA, iPSA, and/or hK2) are received by the at least one processor. The values may be received in any suitable way including, but not limited to, through a local input interface such as a keyboard, touch screen, microphone, or other input device, from a network-connected interface that receives the value(s) from a device located remote from the processor(s), or directly from one or more detectors that measure the blood marker value(s) (e.g., in an implementation where the processor(s) are integrated with a measurement device that includes the one or more detectors).

In response to receiving patient data value(s) and blood marker values, the process proceeds to step 103, where at least one predictive model (e.g., a logistic regression model) is evaluated to determine a likelihood of prostate cancer pathology associated with adverse outcomes, wherein the likelihood is based, at least in part (e.g., solely), on the received patient data value(s) and received blood marker values. As described in detail herein, one or more clinical factors (e.g., prostate volume on prior biopsy) may optionally be used in determining a particular predictive model to use and/or may be used as input values to evaluate a selected model.

After determining a likelihood of prostate cancer pathology associated with adverse outcomes, the process proceeds to step 104, where the probability is outputted or communicated to a user (e.g., a physician, a healthcare provider, and/or a patient) to guide further diagnostic procedure and/or treatment decisions.

Other aspects of the disclosure provide computer implemented methods for determining the likelihood that a prostate tissue sample obtained from the subject through radical prostatectomy would contain aggressive prostate cancer.

Such methods may involve receiving, via an input interface, information indicative of the level of protein markers (e.g., tPSA, fPSA, iPSA, and/or hK2) present in a sample (e.g., a blood sample) of a subject and receiving, via an input interface, patient information, such as information relating to the subject's age, prostate volume, and/or total tumor length. In some embodiments, the methods further involve evaluating, using at least one processor, a suitable predictive model (e.g., a logistic regression model) based, at least in part, on the received information to determine a likelihood of aggressive. The predictive model may generate the likelihood of aggressive prostate cancer based, at least in part, on measured levels of tPSA, fPSA, iPSA, and/or hK2 and patient information, such as information relating to the subject's age prostate volume, and/or total tumor length.

The probability may be outputted or communicated in any suitable way. For example, in some embodiments, the probability may be outputted or communicated by displaying a numeric value representing the probability on a display screen of a device. In other embodiments, the probability may be outputted or communicated using one or more lights or other visual indicators on a device. In yet other embodiments, the probability may be provided or communicated using audio output, tactile output, visual output, or some combination of one or more of audio, tactile, and visual output. In some embodiments, outputting or communicating the probability comprises sending information to a network-connected device to inform a user (e.g., a doctor, a healthcare provider, and/or a patient) about the determined probability. For example, the probability may be determined by one or more processors located at a remote site, and an indication of the probability may be sent to an electronic device of a user (e.g., a physician, a healthcare provider, or a patient) using one or more networks, in response to determining the probability at the remote site. The electronic device that provides output to a user in accordance with the techniques described herein may be any suitable device including, but not limited to, a laptop, desktop, or tablet computer, a smartphone, a pager, a personal digital assistant, and an electronic display.

In some embodiments, the probability of the event associated with prostate cancer is determined in accordance with equation (1), reproduced below:

$$Probability = \frac{{e}^{L}}{1 + {e}^{L}} \qquad ( 1 )$$

where the logit (L) is determined using any of a plurality of logistic regression models. Non-limiting examples of different types of logistic regression models that may be used in accordance with the techniques described herein include:
1. Simple Model (tPSA only)

L=β₀+β₁(Age)+β₂(tPSA)+ (2)

or

L=β₀+β₁tpsa (3)

### 2. Four Assay Model Using Free/Total Ratio

In this model, the ratio of free PSA to total PSA is substituted for the free PSA term.

$$L = {β}_{0} + {β}_{1} ( Age ) + {β}_{2} ( tPSA ) + {β}_{3} ( \frac{fPSA}{tPSA} ) + {β}_{4} ( iPSA ) + {β}_{5} ( hK 2 ) \qquad ( 4 )$$

### 3. Four Assay Model Using Log(tPSA) and Free/Total Ratio

In this model, the log of tPSA is substituted for the tPSA term to account for the increased contribution of this predictive factor.

$$L = {β}_{0} + {β}_{1} ( Age ) + {β}_{2} ( log [ tPSA ] ) + {β}_{3} ( \frac{fPSA}{tPSA} ) + {β}_{4} ( iPSA ) + {β}_{5} ( hK 2 ) ) \qquad ( 5 )$$

### 4. Polynomial Model

In this model, additional non-linear terms for tPSA and fPSA are included. In the example equation provided below, the square of tPSA is used to emphasize the direct relationship between this term and risk of prostate cancer, and the square root of the free/total PSA term is used to reflect the inverse association of this term with risk. It should be appreciated however, that polynomial terms of higher order (e.g., cubic) may also be included in some embodiments.

$$L = {β}_{0} + {β}_{1} ( Age ) + {β}_{2} ( tPSA ) + {β}_{3} ( fPSA ) + {β}_{4} ( iPSA ) + {β}_{5} ( hK 2 ) + {β}_{6} ( {tPSA}^{2} ) + {β}_{7} ( \sqrt{\frac{fPSA}{tPSA}} ) ) \qquad ( 6 )$$

### 5. Linear Splines for all Four Assays

In this model, linear splines are added, with a single knot at the median value. The splines may be determined using the following equations:

sp1(x)=x if x<knot

sp1(x)=knot if x≥knot

sp2(x)=0 if x<knot

sp2(x)=x−knot if x≥knot (7)

with the model being represented as:

L=β₀+β₁(Age)+β₂(tPSA)+β₃(fPSA)+β₄(iPSA)+β₅(hK2)+β₆(sp1[tPSA])+β₇(sp2[tPSA])+β₈(sp1[fPSA])+β₉(sp2[fPSA])+β₁₀(sp1[iPSA])+β₁₁(sp2[iPSA])++β₁₂(sp1[hK2])+β₁₃(sp2[hK2]) (8)

6. Linear Splines for tPSA and fPSA

In this model, linear splines are included only for tPSA and fPSA to reduce the number of variables and simplify the model.

L=β₀+β₁(Age)+β₂(tPSA)+β₃(fPSA)+β₄(iPSA)+β₅(hK2)+β₆(sp1[tPSA])+β₇(sp2[tPSA])+β₈(sp1[fPSA])+β₉(sp2[fPSA]) (9)

In the equations above “priorbx” is a binary value indicate of whether a subject had a prior biopsy to detect prostate cancer. A value of 1 indicates that a prior biopsy occurred and a value of 0 indicates that the prior biopsy did not occur.

### 7. Cubic Splines for all Four Assays

In this model, cubic splines are included for each term. In the example provided below, a cubic spline with four knots is described. It should be appreciated, however, that a cubic spline using any suitable number of knots including, but not limited to, five knots, six knots, seven knots, and eight knots, may alternatively be used. The splines may be determined using the following equations:

$$\begin{matrix}sp [ x ] 1 = {max ( [ x ] - knot 1 , 0 )}^{3} - {max ( [ x ] - knot 3 , 0 )}^{3} \frac{knot 4 - knot 1}{knot 4 - \text{?}} + {max ( [ x ] - knot 4 , 0 )}^{3} \frac{knot 3 - knot 1}{knot 4 - knot 3} & ( 10 ) \\ sp [ x ] 2 = {max ( [ x ] - knot 2 , 0 )}^{3} - {max ( [ x ] - knot 3 , 0 )}^{3} \frac{knot 4 - knot 2}{knot 4 - knot 3} + {max ( [ x ] - knot 2 , 0 )}^{3} \frac{knot 3 - knot 2}{knot 4 - knot 3} \\ \text{?} \text{indicates text missing or illegible when filed} & ( 11 )\end{matrix}$$

where knot1 and knot4 are external knots for the cubic spline, and knot2 and knot3 are internal knots for the cubic spline. The external knots may be set as the minimum and maximum levels of tPSA, fPSA, iPSA, and/or hK2 in a population. An internal knot (e.g., knot2) may be set as the 33.3 percentile value of tPSA, fPSA, iPSA, and/or hK2 levels in a population. Another internal knot (e.g., knot3) may be set as the 66.6 percentile value of tPSA, fPSA, iPSA, and/or hK2 levels in a population.

In some embodiments, the internal knots are specified within the range of between about 2 to about 8 and between about 3 to about 6 for tPSA, between about 0.25 to about 2 and between about 0.5 to about 1.5 for fPSA, between about 0.2 to about 0.5 and between about 0.4 to about 0.8 for iPSA, and between about 0.02 to about 0.04 and between about 0.04 to about 0.08 for hK2. For example, in one implementation, values of 3.92 and 5.61 are used for the internal knots for tPSA, values of 0.82 and 1.21 are used for the internal knots for fPSA, values of 0.3 and 0.51 are used for the internal knots of iPSA, and values of 0.036 and 0.056 are used for the internal knots of hK2.

In certain embodiments, one or more internal knots for tPSA may independently be in the range of between about 3 to about 5, between about 3 to about 6, between about 2.5 to about 6, between about 2.5 to about 6.5, between about 5 to about 8, between about 5.5 to about 8, between about 5 to about 9, between about 5 to about 10, between about 1 to about 5, between about 1 to about 4, and between about 1 to about 3. Other ranges are also possible.

In certain embodiments, one or more internal knots for fPSA may independently be in the range of between about 0.1 to about 1.0, between about 0.1 to about 1.2, between about 0.3 to about 0.8, between about 0.4 to about 0.9, between about 0.5 to about 1.2, between about 0.7 to about 1.4, between about 0.7 to about 0.9, between about 1.1 to about 1.6, between about 1.1 to about 1.2, and between about 1.1 to about 2. Other ranges are also possible.

In certain embodiments, one or more internal knots for iPSA may independently be in the range of between about 0.05 to about 0.5, between about 0.1 to about 0.5, between about 0.2 to about 0.5, between about 0.1 to about 0.8, between about 0.2 to about 0.8, between about 0.4 to about 0.8, between about 0.4 to about 1.0, between about 0.3 to about 0.6, between about 0.5 to about 1.0, and between about 0.6 to about 0.8. Other ranges are also possible.

In certain embodiments, one or more internal knots for hK2 may independently be in the range of between about 0.01 to about 0.03, between about 0.01 to about 0.04, between about 0.01 to about 0.05, between about 0.02 to about 0.05, between about 0.02 to about 0.06, between about 0.03 to about 0.05, between about 0.4 to about 0.07, between about 0.04 to about 1.0, between about 0.5 to about 1.0, and between about 0.6 to about 1.0. Other ranges are also possible.

As discussed above, cubic splines incorporating any suitable number of internal knots (e.g., three, four, five, six internal knots) may be used, and the example of a cubic spline including two internal knots is provided merely for illustration and not limitation. In embodiments that include more than two internal knots, the knots may be placed within one or more of the ranges discussed above, or in some other suitable range. For example, in some embodiments, the knots may be specified such that the length of the segments of the spline between each of the pairs of neighboring knots is essentially equal.

The model may be represented as:

L=β₀+β₁(Age)+β₂(tPSA)+β₃(fPSA)+β₄(iPSA)+β₅(hK2)+β₆(sp1[tPSA])+β₇(sp2[tPSA])+β₈(sp1[fPSA])+β₉(sp2[fPSA])+β₁₀(sp1[iPSA])+β₁₁(sp2[iPSA])+β₁₂(sp1[hK2])+β₁₃(sp2[hK2]) (12)

The spline terms of sp1(tPSA), sp2(tPSA), sp1(fPSA), and sp2(fPSA) in the model above may be determined according to the cubic spline formula presented above under model #7 above (Equations (10 and 11)).

### Computer Implementation

An illustrative implementation of a computer system 106 on which some or all of the techniques and/or user interactions described herein may be implemented is shown in FIG. 1B. The computer system 106 may include one or more processors 107 and one or more computer-readable non-transitory storage media (e.g., memory 108 and one or more non-volatile storage media 110). The processor(s) 107 may control writing data to and reading data from the memory 108 and the non-volatile storage device 110 in any suitable manner, as the aspects of the present invention described herein are not limited in this respect.

To perform any of the functionality described herein, the processor(s) 107 may execute one or more instructions, such as program modules, stored in one or more computer-readable storage media (e.g., the memory 108), which may serve as non-transitory computer-readable storage media storing instructions for execution by the processor 107. Generally, program modules include routines, programs, objects, components, data structures, etc. that perform particular tasks or implement particular abstract data types. Embodiments may also be implemented in distributed computing environments where tasks are performed by remote processing devices that are linked through a communications network. In a distributed computing environment, program modules may be located in both local and remote computer storage media including memory storage devices. Data inputs and program commands may be received by the computer 106 through a input interface 109. The input interface 109 may comprise a keyboard, touchscreen, USB port, CD drive, DVD drive, or other input interface.

Computer 106 may operate in a networked environment using logical connections to one or more remote computers. The one or more remote computers may include a personal computer, a server, a router, a network PC, a peer device or other common network node, and typically include many or all of the elements described above relative to the computer 106. Logical connections between computer 106 and the one or more remote computers may include, but are not limited to, a local area network (LAN) and a wide area network (WAN), but may also include other networks. Such networks may be based on any suitable technology and may operate according to any suitable protocol and may include wireless networks, wired networks or fiber optic networks. Such networking environments are commonplace in offices, enterprise-wide computer networks, intranets and the Internet.

When used in a LAN networking environment, the computer 106 may be connected to the LAN through a network interface or adapter. When used in a WAN networking environment, the computer 106 typically includes a modem or other means for establishing communications over the WAN, such as the Internet. In a networked environment, program modules, or portions thereof, may be stored in the remote memory storage device.

Various inputs described herein for assessing a risk of prostate cancer and/or determining a prostate gland volume may be received by computer 106 via a network (e.g., a LAN, a WAN, or some other network) from one or more remote computers or devices that stores data associated with the inputs. One or more of the remote computers/devices may perform analysis on remotely-stored data prior to sending analysis results as the input data to computer 106. Alternatively, the remotely stored data may be sent to computer 106 as it was stored remotely without any remote analysis. Additionally, inputs may be received directly by a user of computer 106 using any of a number of input interfaces (e.g., input interface 109) that may be incorporated as components of computer 106.

Various outputs described herein, including output of a probability of prostate cancer risk and/or prostate gland volume, may be provided visually on an output device (e.g., a display) connected directly to computer 106 or the output(s) may be provided to a remotely-located output device connected to computer 106 via one or more wired or wireless networks, as embodiments of the invention are not limited in this respect. Outputs described herein may additionally or alternatively be provided other than using visual presentation. For example, computer 106 or a remote computer to which an output is provided may include one or more output interfaces including, but not limited to speakers, and vibratory output interfaces, for providing an indication of the output. Any of these outputs may be used to communicate the results of any of the herein described methods to one or more users (e.g., a physician, a healthcare provider, and/or a patient).

It should be appreciated that although computer 106 is illustrated in FIG. 1B as being a single device, in some embodiments, computer 106 may comprise a plurality of devices communicatively coupled to perform some or all of the functionality described herein, and computer 106 is only one illustrative implementation of a computer that may be used in accordance with embodiments of the invention. For example, in some embodiments, computer 106 may be integrated into and/or in electronic communication with a system. As described above, in some embodiments, computer 106 may be included in a networked environment, where information about one or more blood markers, used to determine a probability of prostate cancer, is sent from an external source to computer 106 for analysis using one or more of the techniques described herein. An illustrative networked environment 111 in accordance with some embodiments of the invention is shown in FIG. 1C. In networked environment 111, computer 106 is connected to an assay system 112 via network 114. As discussed above, network 114 may be any suitable type of wired or wireless network, and may include one or more local area networks (LANs) or wide area networks (WANs), such as the Internet.

The calculation methods, steps, simulations, algorithms, systems, and system elements described herein may be implemented using a computer system, such as the various embodiments of computer systems described below. The methods, steps, systems, and system elements described herein are not limited in their implementation to any specific computer system described herein, as many other different machines may be used.

The computer system may include a processor, for example, a commercially available processor such as one of the series x86, Celeron and Pentium processors, available from Intel, similar devices from AMD and Cyrix, the 680X0 series microprocessors available from Motorola, the PowerPC microprocessor from IBM, and ARM processors. Many other processors are available, and the computer system is not limited to a particular processor.

A processor typically executes a program called an operating system, of which Windows 7, Windows 8, UNIX, Linux, DOS, VMS, MacOS and OSX, and iOS are examples, which controls the execution of other computer programs and provides scheduling, debugging, input/output control, accounting, compilation, storage assignment, data management and memory management, communication control and related services. The processor and operating system together define a computer platform for which application programs in high-level programming languages are written. The computer system is not limited to a particular computer platform.

The computer system may include a memory system, which typically includes a computer readable and writeable non-volatile recording medium, of which a magnetic disk, optical disk, a flash memory, and tape are examples. Such a recording medium may be removable, for example, a floppy disk, read/write CD or memory stick, or may be permanent (such as, for example, a hard drive).

Such a recording medium stores signals, typically in binary form (i.e., a form interpreted as a sequence of one and zeros). A disk (e.g., magnetic or optical) has a number of tracks, on which such signals may be stored, typically in binary form, i.e., a form interpreted as a sequence of ones and zeros. Such signals may define a software program, e.g., an application program, to be executed by the microprocessor, or information to be processed by the application program.

The memory system of the computer system also may include an integrated circuit memory element, which typically is a volatile, random access memory such as a dynamic random access memory (DRAM) or static memory (SRAM). Typically, in operation, the processor causes programs and data to be read from the non-volatile recording medium into the integrated circuit memory element, which typically allows for faster access to the program instructions and data by the processor than does the non-volatile recording medium.

The processor generally manipulates the data within the integrated circuit memory element in accordance with the program instructions and then copies the manipulated data to the non-volatile recording medium after processing is completed. A variety of mechanisms are known for managing data movement between the non-volatile recording medium and the integrated circuit memory element, and the computer system that implements the methods, steps, systems and system elements described above is not limited thereto. The computer system is not limited to a particular memory system.

At least part of such a memory system described above may be used to store one or more data structures (e.g., look-up tables) or equations described above. For example, at least part of the non-volatile recording medium may store at least part of a database that includes one or more of such data structures. Such a database may be any of a variety of types of databases, for example, a file system including one or more flat-file data structures where data is organized into data units separated by delimiters, a relational database where data is organized into data units stored in tables, an object-oriented database where data is organized into data units stored as objects, another type of database, or any combination thereof.

The computer system may include a video and audio data I/O subsystem. An audio portion of the subsystem may include an analog-to-digital (A/D) converter, which receives analog audio information and converts it to digital information. The digital information may be compressed using known compression systems for storage on the hard disk to use at another time. A typical video portion of the I/O subsystem may include a video image compressor/decompressor of which many are known in the art. Such compressor/decompressors convert analog video information into compressed digital information, and vice-versa. The compressed digital information may be stored on hard disk for use at a later time.

The computer system may include one or more output devices. Example output devices include a cathode ray tube (CRT) display, liquid crystal displays (LCD) and other video output devices, printers, communication devices such as a modem or network interface, storage devices such as disk or tape, and audio output devices such as a speaker.

The computer system also may include one or more input devices. Example input devices include a keyboard, keypad, track ball, mouse, pen and tablet, communication devices such as described above, and data input devices such as audio and video capture devices and sensors. The computer system is not limited to the particular input or output devices described herein.

It should be appreciated that one or more of any type of computer system may be used to implement various embodiments described herein. Aspects of the disclosure may be implemented in software, hardware or firmware, or any combination thereof. The computer system may include specially programmed, special purpose hardware, for example, an application-specific integrated circuit (ASIC). Such special-purpose hardware may be configured to implement one or more of the methods, steps, simulations, algorithms, systems, and system elements described above as part of the computer system described above or as an independent component.

The computer system and components thereof may be programmable using any of a variety of one or more suitable computer programming languages. Such languages may include procedural programming languages, for example, C, Pascal, Fortran and BASIC, object-oriented languages, for example, C++, Java, Eiffel, and other languages, such as a scripting language or even an assembly language.

The methods, steps, simulations, algorithms, systems, and system elements may be implemented using any of a variety of suitable programming languages, including procedural programming languages, object-oriented programming languages, other languages and combinations thereof, which may be executed by such a computer system. Such methods, steps, simulations, algorithms, systems, and system elements can be implemented as separate modules of a computer program, or can be implemented individually as separate computer programs. Such modules and programs can be executed on separate computers.

Such methods, steps, simulations, algorithms, systems, and system elements, either individually or in combination, may be implemented as a computer program product tangibly embodied as computer-readable signals on a computer-readable medium, for example, a non-volatile recording medium, an integrated circuit memory element, or a combination thereof. For each such method, step, simulation, algorithm, system, or system element, such a computer program product may comprise computer-readable signals tangibly embodied on the computer-readable medium that define instructions, for example, as part of one or more programs, that, as a result of being executed by a computer, instruct the computer to perform the method, step, simulation, algorithm, system, or system element.

It should be appreciated that various embodiments may be formed with one or more of the above-described features. The above aspects and features may be employed in any suitable combination as the present invention is not limited in this respect. It should also be appreciated that the drawings illustrate various components and features which may be incorporated into various embodiments. For simplification, some of the drawings may illustrate more than one optional feature or component. However, the invention is not limited to the specific embodiments disclosed in the drawings. It should be recognized that the disclosure encompasses embodiments which may include only a portion of the components illustrated in any one drawing figure, and/or may also encompass embodiments combining components illustrated in multiple different drawing figures.

The embodiments and methods described herein may be combined with one or more embodiments and/or methods described in U.S. application Ser. No. 14/671,355, filed on Mar. 27, 2015 and entitled “Compositions and Methods Related to Diagnosis of Prostate Cancer”, and published as US20160025732A1, which is hereby incorporated by reference in its entirety for all purposes.

### Treatment of Prostate Cancer

A subject at risk for prostate cancer pathology associated with adverse outcomes or aggressive prostate cancer, as identified using the methods described herein, may be treated with any appropriate therapeutic agent or any combination of appropriate therapies. In some embodiments, provided methods may include selecting a treatment for a subject based on the output of the described method, e.g., determining a likelihood of prostate cancer pathology associated with adverse outcomes. In some embodiments, provided methods may include selecting a treatment for a subject based on the output of the described method determining a likelihood of aggressive prostate cancer.

In some embodiments, the method comprises one or both of selecting or administering a therapeutic agent, e.g., a chemotherapy, a radiation therapy, a surgical therapy, a cryotherapy, a hormone therapy, and/or an immunotherapy, for administration to the subject based on the output of the assay, e.g., determining a likelihood of prostate cancer pathology associated with adverse outcomes. In some embodiments, the method comprises one or both of selecting or administering a therapeutic agent, e.g., a chemotherapy, a radiation therapy, a surgical therapy, a cryotherapy, a hormone therapy, and/or an immunotherapy, for administration to the subject based on the output of the assay determining a likelihood of aggressive prostate cancer.

In some embodiments, the therapeutic agent is administered one or more times to the subject. The therapeutic agent, e.g., chemotherapy, radiation therapy, surgical therapy, cryotherapy, hormone therapy, and/or immunotherapy, may be administered along with another therapy as part of a combination therapy for treatment of the prostate cancer. Combination therapy, e.g., chemotherapy and radiation therapy, may be provided in multiple different configurations. The first therapy may be administered before or after the administration of the other therapy. In some situations, the first therapy and another therapy (e.g., a therapeutic agent) are administered concurrently, or in close temporal proximity (e.g., a short time interval between the therapies, such as during the same treatment session). The first agent and the other therapy may also be administered at greater temporal intervals.

In some embodiments, a chemotherapeutic agent is administered to a subject. Examples of the chemotherapeutic agents include, but are not limited to, Docetaxel (Taxotere), Cabazitaxel (Jevtana), Mitoxantrone (Novantrone), and Estramustine (Emcyt).

In some embodiments, a radiation therapy is administered to a subject. Examples of radiation therapy include, but are not limited to, ionizing radiation, gamma-radiation, neutron beam radiotherapy, electron beam radiotherapy, proton therapy, brachytherapy, systemic radioactive isotopes, and radiosensitizers.

In some embodiments, a surgical therapy is administered to a subject. Examples of a surgical therapy include, but are not limited to, radical prostatectomy, radical retropubic prostatectomy, radical perineal prostatectomy, laparoscopic radical prostatectomy, and robotic-assisted laparoscopic radical prostatectomy.

In some embodiments, a hormone therapy is administered to a subject. Examples of a hormone therapy include, but are not limited to, orchiectomy, luteinizing hormone-releasing hormone (LHRH) agonists (e.g., Leuprolide, Goserelin, Triptorelin, and Histrelin), LHRH antagonists (e.g., Degarelix), CYP17 inhibitors (e.g., Abiraterone), and anti-androgens (e.g., Flutamide, Bicalutamide, Nilutamide, Enzalutamide, Estrogen, and Ketoconazole).

In some embodiments, a cryotherapy is administered to a subject. In some embodiments, an immunotherapy is administered to a subject. In some embodiments, the immunotherapy is sipuleucel-T. In some embodiments, an anti-metastasis therapy is administered to a subject. Examples of an anti-metastasis therapy include, but are not limited to, bisphosphonates (e.g., Zoledronic acid), Denosumab, and corticosteroids (e.g., prednisone and dexamethasone).

Without further elaboration, it is believed that one skilled in the art can, based on the above description, utilize the present disclosure to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the methods, compositions, and systems provided herein and are not to be construed in any way as limiting their scope.

### Example 1: Four Kallikrein Markers and Age were Significantly Associated with the Likelihood of a Tissue Sample Obtained from RP Containing Prostate Cancer Pathology Associated with Adverse Outcomes in Patients Diagnosed with Low-Grade Cancer on Biopsy

### Patient Population

A recent large, US multi-center prospective trial enrolled 1312 men referred for prostate biopsy for suspicion of prostate cancer regardless of age, PSA, digital rectal exam findings, or prior biopsy status. A subgroup of men, who were found to have low-grade (Gleason 6) cancer on biopsy and underwent radical prostatectomy (RP), was selected to determine whether certain pre-biopsy information and post-biopsy information were associated with certain prostate cancer grades in the surgical specimen. 177 serum samples from men with biopsy-detected Gleason 6 PCa who underwent RP from 2003 to 2013 and had a serum specimen in the institution's bio-repository were identified. Inclusion criteria were age 45-75 and total PSA 1.5-15.0 ng/ml. Kallikrein (i.e., tPSA, fPSA, iPSA, and hK2) levels were calculated for all men.

### Laboratory Methodology

All phlebotomy samples were handled and shipped according to a standardized study protocol to OPKO Lab in Nashville, Tenn. for four-kallikrein testing.

### Statistical Analysis

The primary outcome was prostate cancer pathology associated with adverse outcomes at RP, defined as any Gleason score ≥8 or Gleason 3+4 with ≥pT3b. The kallikrein levels along with other standard clinical and pathologic characteristics were assessed to evaluate their association with cancer associated with adverse outcomes at RP. Variables that showed at least moderate association with the outcome data to include in a multivariate logistic regression model were then identified.

The variables assessed were tPSA, fPSA, iPSA, hK2, f/tPSA ratio, results of a digital rectal examination, occurrence of any negative biopsy since an initial diagnosis of the prostate cancer, number of biopsy cores, number of positive cores on prior biopsy, percent positive cores on prior biopsy, maximum tumor involvement percentage, prostate volume on prior biopsy, and PSA density.

The variable were assessed using bivariate analysis to determine the significance of the association between the variable and cancer having any Gleason score ≥8 at RP or the risk of cancer of having Gleason 3+4 with ≥pT3b.

### Results

Patient demographics and clinical characteristics and their associated p-values are shown in Table 1. Outcome 0 was cancer having any Gleason score <8 at RP and having Gleason 3+4 with <pT3b. Outcome 1 was cancer having any Gleason score ≥8 at RP and having Gleason 3+4 with ≥pT3b.

TABLE 1
Patient Demographic and Clinical Characteristics.

| | | outcome = 0 | | outcome = 1 | | |
|---|---|---|---|---|---|---|
| | Statistic | (N = 413; 96%) | | (N = 15; 3.5%) | | p-value |
| Age | Median (IQR) | 64 | (59, 67) | 65 | (59, 68) | 0.6 |
| Kallikrein markers | Median (IQR) | −0.81 | (−1.45, −0.02) | 0.75 | (−0.70, 1.38) | 0.001 |
| tPSA | Median (IQR) | 5.81 | (4.38, 7.90) | 7.28 | (6.36, 10.32) | 0.014 |
| f/tPSA ratio | Median (IQR) | 0.13 | (0.09, 0.17) | 0.11 | (0.07, 0.17) | 0.2 |
| Prostate volume | Median (IQR) | 49.00 | (37.10, 65.00) | 40.00 | (34.00, 55.00) | 0.2 |
| (cc) (N = 413) | | | | | | |
| PSA Density | Median (IQR) | 0.12 | (0.08, 0.17) | 0.17 | (0.12, 0.26) | 0.017 |
| (N = 413) | | | | | | |
| Abnormal DRE | n (%) | 32 | (7.7%) | 1 | (6.7%) | 1 |
| Had prior negative | n (%) | 70 | (17%) | 4 | (27%) | 0.3 |
| biopsy? | | | | | | |
| Tumor Clinical Stage | | | | | | |
| T1c | n (%) | 369 | (89%) | 14 | (93%) | 0.3 |
| T2a | n (%) | 27 | (6.5%) | 0 | (0%) | |
| T2b | n (%) | 3 | (0.7%) | 1 | (6.7%) | |
| T2c | n (%) | 1 | (0.2%) | 0 | (0%) | |
| n.a. | n (%) | 13 | (3.1%) | 0 | (0%) | |
| Biopsy Cores | Median (IQR) | 10 | (10, 10) | 10 | (10, 10) | 0.4 |
| Positive Cores | Median (IQR) | 2 | (1, 3) | 3 | (1, 4) | 0.3 |
| Positive cores ratio | Median (IQR) | 0.20 | (0.10, 0.30) | 0.30 | (0.10, 0.40) | 0.5 |
| Maximum tumor | Median (IQR) | 6.20 | (4.00, 17.50) | 5.00 | (4.40, 20.00) | 0.8 |
| involvement (%) | | | | | | |
| Total tumor length | Median (IQR) | 1.80 | (0.80, 4.70) | 2.40 | (0.60, 5.90) | 0.8 |
| (mm) (N = 422) | | | | | | |
| Pathology stage | | | | | | |
| pT2a | n (%) | 68 | (16%) | 0 | (0%) | |
| pT2b | n (%) | 1 | (0.2%) | 0 | (0%) | |
| pT2c | n (%) | 310 | (75%) | 6 | (40%) | |
| pT3a | n (%) | 34 | (8.2%) | 1 | (6.7%) | |
| pT3b | n (%) | 0 | (0%) | 8 | (53%) | |
| RP Gleason | | | | | | |
| 3 + 2 | n (%) | 22 | (5.3%) | 0 | (0%) | |
| 3 + 3 | n (%) | 213 | (52%) | 1 | (6.7%) | |
| 3 + 4 | n (%) | 176 | (43%) | 7 | (47%) | |
| 3 + 5 | n (%) | 2 | (0.5%) | 0 | (0%) | |
| 4 + 3 | n (%) | 0 | (0%) | 7 | (47%) | |

Bivariate analysis showed the four kallikrein markers and PSA density were all significantly associated with prostate cancer having Gleason score ≥8 or Gleason 3+4 with ≥pT3b at RP (p≤0.02 for all). f/t PSA ratio, prostate volume, number of biopsy cores, number of positive biopsy cores, positive core ratio, maximum tumor involvement percent, and total tumor length were not associated with upgrade (p≥0.2 for all).

A formula for a predictive model for calculating risk of cancer having any Gleason score ≥8 at RP or Gleason 3+4 with ≥pT3b was developed and is presented below. Weighting coefficients are within the ranges specified in Tables herein. The variables of the formulae are described in Table below.

L=β₁₀*[β₀+β₁(Age)+β₂(tPSA)+β₃sp1(tPSA)+β₄sp2(tPSA)+β₅(fPSA)+β₆sp1(fPSA)+β₇sp2(fPSA)+β₈(iPSA)+β₉(hK2)]+β₁₁

| Weighting Coefficient | | |
|---|---|---|
| Ranges | Low | High |
| β₀ | −7.35E+00 | −6.00E+00 |
| B₁ | 4.79E−02 | 6.38E−02 |
| B₂ | 7.44E−01 | 9.19E−01 |
| B₃ | −6.43E−03 | −4.32E−03 |
| B₄ | 1.20E−02 | 1.66E−02 |
| B₅ | −6.27E+00 | −4.43E+00 |
| B₆ | 7.63E−01 | 1.04E+00 |
| B₇ | −2.76E+00 | −2.17E+00 |
| B₈ | 1.96E+00 | 2.40E+00 |
| B₉ | 6.62E+00 | 7.59E+00 |
| B₁₀ | 5.00E−01 | 4.00E+00 |
| B₁₁ | −6.00E+00 | −1.00E+00 |

$$Adverse Outcome = \frac{{e}^{X β}}{1 + {e}^{X β}} \qquad ( 15 )$$

### Restricted Cubic Spline Terms:

For some variables in the models (total PSA and free PSA), restricted cubic spline terms were included, meaning that two additional terms are added to each of the models for each splined term. The formulas for calculating the two spline terms are below.

$$\begin{matrix}sp [ var ] 1 = {max ( [ var ] - knot 1 , 0 )}^{3} - {max ( [ var ] - knot 3 , 0 )}^{3} \frac{knot 4 - knot 1}{knot 4 - knot 3} + {max ( [ var ] - knot 4 , 0 )}^{3} \frac{knot 3 - knot 1}{knot 4 - knot 3} & ( 10 ) \\ sp [ var ] 2 = {max ( [ var ] - knot 2 , 0 )}^{3} - {max ( [ var ] - knot 3 , 0 )}^{3} \frac{knot 4 - knot 2}{knot 4 - \text{?}} + {max ( [ var ] - knot 4 , 0 )}^{3} \frac{knot 3 - knot 2}{knot 4 - knot 3} \\ \text{?} \text{indicates text missing or illegible when filed} & ( 11 )\end{matrix}$$

Sp[var]1 and sp[var]2 are computed for total and free PSA using the formulae above. The spline term for total PSA was calculated using knot values within the ranges specified in Table 3.

TABLE 3
Variables for formula for calculating risk of prostate
cancer pathology associated with adverse outcomes.

| Variable Name | Description |
|---|---|
| age | Age at Blood Draw |
| tpsa | Total PSA in ng/ml |
| fpsa | Free PSA in ng/ml |
| ipsa | Intact PSA in ng/ml |
| hk2 | hK2 in ng/ml |
| sptpsa1 | First spline term for total PSA |
| sptpsa2 | Second spline term for total PSA |
| spfpsa1 | First spline term for free PSA |
| spfpsa2 | Second spline term for free PSA |

The logistic regression algorithm incorporating the blood levels of these four kallikrein markers as well as age demonstrated a higher positive predictive value for prostate cancer having Gleason score ≥8 or Gleason 3+4 with ≥pT3b than any other combination of variables. The logistic regression algorithm had an AUC of 0.745.

The AUC of the algorithm is much larger than the AUC of a base model that includes age and tPSA. This difference is not statistically significant, likely due to the relatively small number of positive outcomes.

Outcome of Gleason Group 3 or Gleason Group 2 with Pathology Stage≥pT3b

| Model | AUC (p = 0.4779) |
|---|---|
| Algorithm model (including 4K panel) | 0.7446 |
| Alternative model (including age and tPSA) | 0.7022 |

### Discussion

This example shows that the logistic regression algorithm incorporating the blood levels of four kallikrein markers as well as age can be a helpful tool for predicting the presence of prostate cancer pathology associated with adverse outcomes in patients who are diagnosed with low-grade disease and are contemplating active surveillance. Among a group of patients with low-grade prostate cancer on biopsy of the prostate, the logistic regression algorithm was associated with prostate cancer pathology associated with adverse outcomes. Thus, the logistic regression algorithm may be beneficial for selecting patients that can safely monitor their cancer versus those who need immediate treatment.

### Example 2: Four Kallikrein Markers and Age were Significantly Associated with Aggressive Prostate Cancer at RP in Patients Diagnosed with Low-Grade Cancer on Biopsy

### Patient Population

A recent large, US multi-center prospective trial enrolled 1312 men referred for prostate biopsy for suspicion of prostate cancer regardless of age, PSA, digital rectal exam findings, or prior biopsy status. A subgroup of men, who were found to have low-grade (Gleason 6) cancer on biopsy and underwent radical prostatectomy (RP), was selected to determine whether certain pre-biopsy information and post-biopsy information were associated with certain prostate cancer grades in the surgical specimen. 177 serum samples from men with biopsy-detected Gleason 6 PCa who underwent RP at Martini Klinik from 2003 to 2013 and had a serum specimen in the institution's bio-repository were identified. Inclusion criteria were age 45-75 and total PSA 1.5-15.0 ng/ml. Kallikrein (i.e., tPSA, fPSA, iPSA, and hK2) levels were calculated for all men.

### Laboratory Methodology

All phlebotomy samples were handled and shipped according to a standardized study protocol to OPKO Lab in Nashville, Tenn. for four-kallikrein testing.

### Statistical Analysis

The primary outcome was aggressive cancer at RP, defined as any Gleason score ≥8 or Gleason 3+4 with ≥pT3a. The kallikrein levels along with other standard clinical and pathologic characteristics were assessed to evaluate their association with aggressive cancer at RP. Variables that showed at least moderate association with the outcome data to include in a multivariate logistic regression model were then identified.

The variables assessed were tPSA, fPSA, iPSA, hK2, f/tPSA ratio, results of a digital rectal examination, occurrence of any negative biopsy since an initial diagnosis of the prostate cancer, number of biopsy cores, number of positive cores on prior biopsy, percent positive cores on prior biopsy, maximum tumor involvement percentage, prostate volume on prior biopsy, and PSA density.

The variable were assessed using bivariate analysis to determine the significance of the association between the variable and cancer having any Gleason score ≥8 at RP or the risk of cancer of having Gleason 3+4 with ≥pT3a.

### Results

Patient demographics and clinical characteristics and their associated p-values are shown in Table 4. Outcome 0 was cancer having any Gleason score <8 at RP and having Gleason 3+4 with <pT3a. Outcome 1 was cancer having any Gleason score ≥8 at RP and having Gleason 3+4 with ≥pT3a.

TABLE 4
Patient Demographic and Clinical Characteristics.

| | | upgrade = 0 | | upgrade = 1 | | |
|---|---|---|---|---|---|---|
| | Statistic | (N = 379; 89%) | | (N = 49; 11%) | | p-value |
| Age | Median (IQR) | 64 | (59, 67) | 64 | (58, 67) | 0.9 |
| Kallikrein | Median (IQR) | −0.88 | (−1.48, −0.12) | −0.07 | (−0.70, 0.66) | <0.0001 |
| markers | | | | | | |
| tPSA | Median (IQR) | 5.70 | (4.36, 7.97) | 7.09 | (5.43, 7.93) | 0.026 |
| f/tPSA ratio | Median (IQR) | 0.13 | (0.09, 0.17) | 0.11 | (0.07 ,0.14) | 0.010 |
| Prostate volume | Median (IQR) | 50.00 | (38.00, 65.00) | 40.00 | (32.00, 51.00) | 0.001 |
| (cc) (N = 413) | | | | | | |
| PSA Density | Median (IQR) | 0.11 | (0.08, 0.16) | 0.16 | (0.11, 0.22) | <0.0001 |
| (N = 413) | | | | | | |
| Abnormal DRE | n (%) | 29 | (7.7%) | 4 | (8.2%) | 0.8 |
| Had prior | n (%) | 67 | (18%) | 7 | (14%) | 0.6 |
| negative biopsy? | | | | | | |
| Tumor Clinical Stage | | | | | | |
| T1c | n (%) | 339 | (89%) | 44 | (90%) | 0.8 |
| T2a | n (%) | 24 | (6.3%) | 3 | (6.1%) | |
| T2b | n (%) | 3 | (0.8%) | 1 | (2.0%) | |
| T2c | n (%) | 1 | (0.3%) | 0 | (0%) | |
| n.a. | n (%) | 12 | (3.2%) | 1 | (2.0%) | |
| Biopsy Cores | Median (IQR) | 10 | (10, 10) | 10 | (10, 10) | 0.5 |
| Positive Cores | Median (IQR) | 2 | (1, 3) | 3 | (2, 4) | 0.002 |
| Positive cores ratio | Median (IQR) | 0.20 | (0.10, 0.30) | 0.25 | (0.20, 0.38) | 0.010 |
| Maximum tumor | Median (IQR) | 5.00 | (3.50, 14.00) | 15.60 | (5.00, 27.80) | 0.0002 |
| involvement (%) | | | | | | |
| Total tumor length | Median (IQR) | 1.70 | (0.70,4.20) | 3.80 | (1.30, 9.15) | 0.001 |
| (mm) (N = 422) | | | | | | |
| Pathology stage | | | | | | |
| pT2a | n (%) | 68 | (18%) | 0 | (0%) | <0.0001 |
| pT2b | n (%) | 1 | (0.3%) | 0 | (0%) | |
| pT2c | n (%) | 310 | (82%) | 6 | (12%) | |
| pT3a | n (%) | 0 | (0%) | 35 | (71%) | |
| pT3b | n (%) | 0 | (0%) | 8 | (16%) | |
| RP Gleason | | | | | | |
| 3 + 2 | n (%) | 21 | (5.5%) | 1 | (2.0%) | <0.0001 |
| 3 + 3 | n (%) | 204 | (54%) | 10 | (20%) | |
| 3 + 4 | n (%) | 152 | (40%) | 31 | (63%) | |
| 3 + 5 | n (%) | 2 | (0.5%) | 0 | (0%) | |
| 4 + 3 | n (%) | 0 | (0%) | 7 | (14%) | |

Bivariate analysis showed the four kallikrein markers, f/t PSA ratio, prostate volume, number of positive cores, positive cores ratio, total tumor length, maximum tumor involvement percentage, and PSA density were all significantly associated with prostate cancer having Gleason score ≥8 or Gleason 3+4 with ≥pT3a at RP (p≤0.02 for all). All other variables were not associated with cancer having Gleason score ≥8 or Gleason 3+4 with ≥pT3a at RP (p≤0.02 for all) (p≥0.2 for all). On multivariate modeling, the four kallikreins, prostate volume, and total tumor length were independently significant as shown in Table 5.

TABLE 5
Multivariate modeling results.

| Variable | p value |
|---|---|
| kallikrein panel | <0.001 |
| Prostate volume | 0.01 |
| Total tumor length | 0.012 |

The kallikrein panel is logit containing the sum of terms associated with tPSA, fPSA, iPSA, hK2, and age.

A formula for a predictive model for calculating risk of cancer having any Gleason score≥8 at RP or Gleason 3+4 with ≥pT3a was developed and is presented below. Weighting coefficients are within the ranges specified in Tables below herein. The variables of the formulae are described in Table below.

L=β₁₂*[β₀+β₁(Age)+β₂(tPSA)+β₃sp1(tPSA)+β₄sp2(tPSA)+β₅(fPSA)+β₆sp1(fPSA)+β₇sp2(fPSA)+β₈(iPSA)+β₉(hK2)]+β₁₀(volume)+β₁₁(tumor_length)+β₁₃

| Weighting Coefficient | | |
|---|---|---|
| Ranges | Low | Hight |
| β₀ | −7.35E+00 | −6.00E+00 |
| B₁ | 4.79E−02 | 6.38E−02 |
| B₂ | 7.44E−01 | 9.19E−01 |
| B₃ | −6.43E−03 | −4.32E−03 |
| B₄ | 1.20E−02 | 1.66E−02 |
| B₅ | −6.27E+00 | −4.43E+00 |
| B₆ | 7.63E−01 | 1.04E+00 |
| B₇ | −2.76E+00 | −2.17E+00 |
| B₈ | 1.96E+00 | 2.40E+00 |
| B₉ | 6.62E+00 | 7.59E+00 |
| B₁₀ | −2.44E−01 | 1.74E−01 |
| B₁₁ | 5.00E−01 | 2.00E+00 |
| B₁₂ | 5.00E−01 | 2.50E+00 |
| B₁₃ | −3.00E+00 | 2.00E+00 |

$$Risk of Aggressive Cancer = \frac{{e}^{X β}}{1 + {e}^{X β}} \qquad ( 15 )$$

### Restricted Cubic Spline Terms:

For some variables in the models (total PSA and free PSA), restricted cubic spline terms were included, meaning that two additional terms are added to each of the models for each splined term. The formulas for calculating the two spline terms are below.

$$\begin{matrix}sp [ var ] 1 = {max ( [ var ] - knot 1 , 0 )}^{3} - {max ( [ var ] - knot 3 , 0 )}^{3} \frac{knot 4 - knot 1}{knot 4 - knot 3} + {max ( [ var ] - knot 4 , 0 )}^{3} \frac{knot 3 - knot 1}{knot 4 - knot 3} & ( 10 ) \\ sp [ var ] 2 = {max ( [ var ] - knot 2 , 0 )}^{3} - {max ( [ var ] - knot 3 , 0 )}^{3} \frac{knot 4 - knot 2}{knot 4 - \text{?}} + {max ( [ var ] - knot 4 , 0 )}^{3} \frac{knot 3 - knot 2}{knot 4 - knot 3} \\ \text{?} \text{indicates text missing or illegible when filed} & ( 11 )\end{matrix}$$

Sp[var]1 and sp[var]2 are computed for total and free PSA using the formulae above. The spline term for total PSA was calculated using knot values within the ranges specified in Table 3.

TABLE
Variables for formula for calculating
risk of aggressive prostate cancer.

| Variable Name | Description |
|---|---|
| age | Age at Blood Draw |
| tpsa | Total PSA in ng/ml |
| fpsa | Free PSA in ng/ml |
| ipsa | Intact PSA in ng/ml |
| hk2 | hK2 in ng/ml |
| sptpsa1 | First spline term for total PSA |
| sptpsa2 | Second spline term for total PSA |
| spfpsa1 | First spline term for free PSA |
| spfpsa2 | Second spline term for free PSA |
| volume | Prostate volume |
| tumor_length | Total tumor length |

The logistic regression algorithm incorporating the blood levels of these four kallikrein markers as well as age demonstrated a higher positive predictive value for prostate cancer having Gleason score ≥8 or Gleason 3+4 with ≥pT3a than any other combination of variables. The logistic regression algorithm had an AUC of 0.7467.

### Discussion

This example shows that the logistic regression algorithm incorporating the blood levels of four kallikrein markers as well as age can be a helpful tool for predicting the presence of aggressive cancer in patients who are diagnosed with low-grade disease and are contemplating active surveillance. Among a group of patients with low-grade prostate cancer on biopsy of the prostate, the logistic regression algorithm was associated with aggressive cancer. Thus, the logistic regression algorithm may be beneficial for selecting patients that can safely monitor their cancer versus those who need immediate treatment.

The AUC of the algorithm is much larger than the AUC of a base model that includes age, tPSA, prostate volume and biopsy total tumor length, and this difference is statistically significant (p<0.05).
Outcome of Gleason Group 3 or Gleason Group 2 with pathology stage≥pT3a

| Model | AUC (p = 0.0455) |
|---|---|
| Algorithm model (including 4K panel, | 0.7467 |
| prostate volume, total tumor length) | |
| Alternative model (including age, tPSA, | 0.7063 |
| prostate volume, total tumor length) | |

### Example 3

The predictive power of the kallikrein panel for adverse outcome was also investigated in the context of using am outcome of biochemical recurrence (by tPSA measurement) after radical prostatectomy, which would indicate poor prognosis for the patient and high risk of developing metastatic prostate cancer. In a cohort of 428 men who underwent radical prostatectomy, 28 were found to have biochemical recurrence.

Multivariate analyses of the candidate predictors of biochemical recurrence reveal that only kallikrein panel was statistically significant in predicting biochemical recurrence. Kaplan-Meier estimator shows clearly that all men with a low kallikrein panel (<20%) have no occurrence of biochemical recurrence in a five year follow up. All occurrences of biochemical recurrence are observed in the group of men with high kallikrein panel (≥20%). Because biochemical recurrence is rare in the Target Population, the cohort size and the number of occurrence of biochemical recurrences is however too small to demonstrate statistical significance (p=0.116). This evidence further demonstrate the capacity of the kallikrein panel (and it constituents: tPSA, fPSA, iPSA, hK2 and age) to correctly identify men at risk of adverse outcome.

We conducted survival analysis using Cox regression to assess the association between 4K panel and the hazard (risk rate) of biochemical recurrence.

The Cox regression with kallikrein panel, prostate volume and total tumor length shows that 4K panel is only significant predictor of biochemical recurrence over time (p=0.019).

| | Hazard | 95% Confidence | |
|---|---|---|---|
| Variable | Ratio | Interval | p-value |
| kallikrein panel | 1.43 | 1.06, 1.93 | 0.019 |
| Prostate volume | 1.00 | 0.98, 1.02 | 0.9 |
| Biopsy total tumor | 0.98 | 0.91, 1.06 | 0.6 |
| length | | | |

We also conducted an alternative model of Cox regression with patients' age and tPSA. Neither patients' age nor tPSA is significantly associate with biochemical recurrence over time (p>0.05 for both).

| | Hazard | 95% Confidence | |
|---|---|---|---|
| Variable | Ratio | Interval | p-value |
| Age | 1.05 | 0.98, 1.14 | 0.2 |
| tPSA | 1.10 | 0.99, 1.22 | 0.086 |

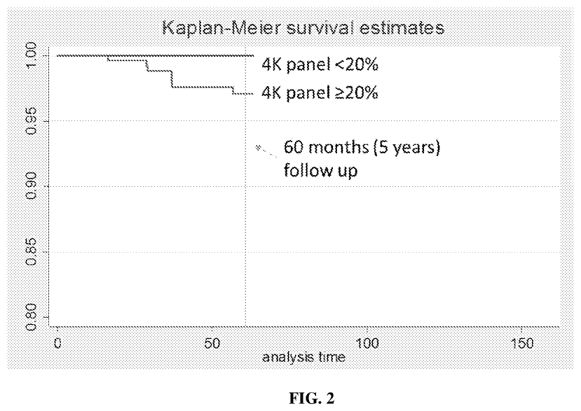

We also constructed a Kaplan-Meier graph, shown in FIG. 2, to compare biochemical recurrence rates between patients with kallikrein panel score lower than 20% and higher 20%. Of patients with kallikrein panel (i.e., 4K panel) score lower than 20%, no one had biochemical recurrence within 5 years of follow-up time.

What is claimed is:

1. An immunoassay-based method of evaluating a subject identified, based on a prostate tissue biopsy, as having prostate cancer characterized by a primary Gleason score of 3, the method comprising:
i) subjecting a blood sample of the subject to immunoassays that measure levels of total prostate specific antigen (tPSA), free prostate specific antigen (fPSA), intact prostate specific antigen (iPSA), and human kallikrein 2 (hK2); and
ii) determining a prostate cancer adverse outcome likelihood score for the subject based on the levels of fPSA, tPSA, iPSA, and hK2 and age of the subject.

2. A method of treating a subject having prostate cancer, wherein the prostate cancer was characterized by a primary Gleason score of 3 based on a biopsy analysis, and wherein the prostate cancer was subsequently determined to be associated with an adverse outcome based on the levels of fPSA, tPSA, iPSA, and hK2 in the subject and age of the subject, the method comprising:
performing a radical prostatectomy procedure to remove the prostate tissue from the subject.

3. A method of evaluating a treatment regimen for a subject identified as having prostate cancer characterized by a primary Gleason score of 3, the method comprising:
(i) obtaining information indicative of a level of total prostate specific antigen (tPSA) and free prostate specific antigen (fPSA), intact prostate specific antigen (iPSA), and human kallikrein 2 (hK2), in the subject;
(ii) determining a prostate cancer adverse outcome likelihood score for the subject based on the levels of fPSA, tPSA, iPSA, and hK2 and age of the subject; and
(iii) determining appropriateness of the treatment regimen based on the likelihood score.

4. The method of claim 3, wherein the treatment regimen is active surveillance.

5. The method of claim 3, wherein the treatment regimen comprises a chemotherapy, a radiation therapy, a surgical therapy, a cryotherapy, a hormone therapy, an immunotherapy, or a combination thereof.

6. The method of any preceding claim, wherein the prostate cancer was identified as having a Gleason score of 3+4.

7. The method of any preceding claim, wherein the measured levels of fPSA, tPSA, iPSA, and hK2 and age of the subject are weighted using a regression model.

8. The method of any preceding claim, wherein determining the likelihood score comprises weighting a cubic spline term based on the measured tPSA level.

9. The method of any preceding claim, wherein determining the likelihood score comprises weighting a cubic spline term based on the measured fPSA level.

10. The method of any preceding claim, further comprising removing at least a portion of the prostate of the subject, wherein the likelihood score is greater than a threshold value.

11. The method of any preceding claim, except claim 2, further comprising treating the subject, wherein treating the subject comprises a chemotherapy, a radiation therapy, a surgical therapy, a cryotherapy, a hormone therapy, an immunotherapy, or a combination thereof, and wherein the likelihood score is greater than a threshold value.

12. The method of any preceding claim, further comprising treating the subject with active surveillance, wherein the likelihood score is less than a threshold value.

13. The method of any one of claims 1 and 4-12, wherein the blood sample is obtained from the subject within 3 months from a biopsy.

14. The method of any preceding claim, wherein step (i) and (ii) are performed within 3 months from a biopsy.

15. The method of any preceding claim further comprising repeating steps (i) and (ii) at least once within 6 months to 12 months from first performing steps (i) and (ii).

16. The method of any preceding claim, further comprising repeating steps (i) and (ii) at least once a year for up to five years.

17. The method of any preceding claim, wherein determining the likelihood score comprises weighting the measured levels of fPSA, tPSA, iPSA, and hK2.

18. The method of any preceding claim, wherein the prostate cancer pathology associated with adverse outcomes has a pathological stage of at least T3b.

19. A method for determining a probability of prostate cancer pathology associated with adverse outcomes, the method comprising:
receiving, via an input interface, information indicative of a level of total prostate specific antigen (tPSA), free prostate specific antigen (fPSA), intact prostate specific antigen (iPSA), and human kallikrein 2 (hK2) in a subject and information indicative of age of a subject;
evaluating, using at least one processor, a logistic regression model based on the received information to determine a probability of prostate cancer pathology associated with adverse outcomes, wherein evaluating the logistic regression model consists essentially of:
determining the probability of prostate cancer pathology associated with adverse outcomes based on the information indicative of the level of tPSA, fPSA, iPSA, and hK2, and the information indicative of the subject's age; and
outputting an indication of the probability of prostate cancer pathology associated with adverse outcomes, wherein the subject has been identified as having Gleason 6 or 3+4 prostate cancer on biopsy.

20. The method of any preceding claim, wherein the model outputs a risk score, wherein the output is indicative of prostate cancer pathology associated with adverse outcomes.

21. The method of any preceding claim, wherein a risk score of less than 7.5% is indicative of low risk prostate cancer pathology associated with adverse outcomes.

22. The method of any preceding claim, wherein a risk score of between 7.5% and 20% is indicative of intermediate risk prostate cancer pathology associated with adverse outcomes.

23. The method of any preceding claim, wherein a risk score of greater than 20% is indicative of high risk prostate cancer pathology associated with adverse outcomes.

24. The method of any preceding claim, wherein the subject is eligible for active surveillance.

25. A computer for determining a probability of prostate cancer pathology associated with adverse outcomes, the computer comprising:
an input interface configured to receive information indicative of a level of total prostate specific antigen (tPSA), free prostate specific antigen (fPSA), intact prostate specific antigen (iPSA), and human kallikrein 2 (hK2) in a subject and information indicative of a subject's age;
at least one processor programmed to evaluate a logistic regression model based, at least in part, on the received information to determine a probability of prostate cancer pathology associated with adverse outcomes, wherein evaluating the logistic regression model consists essentially of:
determining the probability of prostate cancer pathology associated with adverse outcomes, at least in part, on the information indicative of the level of tPSA, fPSA, iPSA, and hK2 and the information indicative of the subject's age; and
an output interface configured to output an indication of the probability of prostate cancer pathology associated with adverse outcomes,
wherein the subject has been identified as having Gleason 6 or 3+4 prostate cancer on biopsy.

26. A system for determining a probability of prostate cancer pathology associated with adverse outcomes, the system comprising:
(a) a detector configured to measure a level of total prostate specific antigen (tPSA), free prostate specific antigen (fPSA), intact prostate specific antigen (iPSA), and human kallikrein 2 (hK2) in a subject; and
(b) a computer in electronic communication with the detector, wherein the computer comprises:
(i) an input interface configured to receive information indicative of a level of tPSA, fPSA, iPSA, and hK2 in the subject and information indicative of the subject's age;
(ii) at least one processor programmed to evaluate a logistic regression model based, at least in part, on the received information to determine a probability of prostate cancer pathology associated with adverse outcomes, wherein evaluating the logistic regression model consists essentially of:
determining the probability of prostate cancer pathology associated with adverse outcomes, at least in part, on the information indicative of the level of tPSA, fPSA, iPSA, and hK2 in the subject and information indicative of the subject's age; and
(iii) an output interface configured to output an indication of the probability of prostate cancer pathology associated with adverse outcomes,
wherein the subject has been identified as having Gleason 6 or 3+4 prostate cancer on biopsy.

27. The method, computer, or system of any preceding claim, wherein a clinical stage of the biopsy is lower than T3.

28. The method, computer, or system of any preceding claim, wherein the subject has been identified as having Gleason 6 and a tPSA level of less than 20 ng/mL.

29. The method, computer, or system of any preceding claim, wherein the subject has been identified as having Gleason 3+4 and a tPSA level of less than 10 ng/mL.

30. A method for determining a probability of at least pT3a prostate cancer, the method comprising:
subjecting a blood sample of the subject to immunoassays that measure levels of total prostate specific antigen (tPSA), free prostate specific antigen (fPSA), intact prostate specific antigen (iPSA), and human kallikrein 2 (hK2) in a subject; and
determining the probability of at least pT3a prostate cancer by weighting the measured levels of fPSA, tPSA, iPSA, and hK2 and information indicative of at least one clinical factor of the subject, wherein the subject has been identified as having Gleason 6 or 3+4 prostate cancer on biopsy.

31. An immunoassay method comprising:
i) subjecting a blood sample of a prostate cancer patient having a primary Gleason score of 3 and a Grade Group designation of 1 or 2 to immunoassays that measure levels of total prostate specific antigen (tPSA), free prostate specific antigen (fPSA), intact prostate specific antigen (iPSA), and human kallikrein 2 (hK2); and
ii) determining a risk score predictive of an underlying adverse pathology associated with adverse outcomes in said prostate cancer patient based on the measured levels of fPSA, tPSA, iPSA, and hK2 and age of the subject, wherein the risk score is provided in a percentage range of between 0-100% and further wherein a risk score of about 0-7.5% is predictive of a low risk of having an underlying adverse pathology; a risk score of about 7.5 to 20% is predictive of an elevated intermediate risk of having an underlying adverse pathology and a risk score of about 20% or greater is predictive of a high risk of having an underlying adverse pathology.

32. The immunoassay method according to claim 31, wherein the prostate cancer patient is on active surveillance as defined by NCCN and AUA.

33. The immunoassay method according to claim 31, wherein the underlying adverse pathology includes seminal vesicle or lymph node invasion.

34. The immunoassay method according to claim 31, wherein the AUC of the method is greater than or equal to about 0.7, greater than or equal to about 0.72, or greater than or equal to about 0.74.
